(19) United States
(12) Patent Application Publication
(10) Pub. No.: US 2010/0312092 A1
(43) Pub. Date: Dec. 9, 2010

(54) Method and system for vascular elastography

(76) Inventors: Roch Listz Maurice, Laval (CA); Guy Cloutier, Repentigny (CA); Jacques Ohayon, Tresserve (FR); Gilles Soulez, Outremont (CA)

Correspondence Address: BCF LLP, 1100 RENE'-LE'VESQUE BLVD. WEST, 25TH FLOOR, MONTREAL, QC, H3B-5C9, CA

(21) Appl. No.: 12/590,715

(22) Filed: Nov. 12, 2009

Related U.S. Application Data
(63) Continuation of application No. 10/588,421, filed on May 17, 2007, now abandoned.

(30) Foreign Application Priority Data
Feb. 9, 2004 (CA) 2457171

(51) Int. Cl. A61B 5/055

(52) U.S. Cl. 600/411

(57) ABSTRACT

The method for vascular elastography comprises: i) obtaining a sequence of radio-frequency (RF) images including pre-tissue-motion and post-tissue-motion images in digital form of a vessel delimited by a vascular wall; the pre-tissue-motion and post-tissue-motion images being representative of first and second time-delayed configuration, of the whole vessel; ii) partitioning both the pre-tissue-motion and post-tissue-motion images within the vascular wall into corresponding data windows; approximating a trajectory between the pre- and post-tissue-motion for corresponding data windows; and using the trajectory for each data window to compute the full strain tensor in each data window, which allow determining the Von Mises coefficient. The method can be adapted for non-invasive vascular elastography (NIVE), for non-invasive vascular micro-elastography (MicroNlVE) on small vessels, and for endovascular elastography (EVE).

### FIELD OF THE INVENTION

The present invention relates to vascular tissue characterization. More specifically, the present invention is concerned with a method and system for vascular elastography imaging.

### BACKGROUND OF THE INVENTION

In the early nineties, Ophir et al., (1991) introduced elastography, which is defined as biological tissue elasticity imaging.

Primary objectives of elastography were to complement B-mode ultrasound as a screening method to detect hard areas in the breast [Garra et al., 1997].

Within the last few years, elastography has also found application in vessel wall characterization using endovascular catheters [Brusseau et al., (2001); de Korte et al., (1997-2000b)]. Indeed, changes in vessel wall elasticity may be indicative of vessel pathologies. It is known, for example, that the presence of plaque stiffens the vascular wall, and that the heterogeneity of its composition may lead to plaque rupture and thrombosis. As indicated below, the mechanical properties of plaques were also recently studied non-invasively.

### A) Non-Invasive Vascular Elastography (NIVE)

Recently, several groups have proposed different approaches to non-invasively characterize superficial arteries by using standard extra-corporal array transducers. Namely, Bang et al. (2003) developed a method to analyze pulsatile motion of carotid artery plaque from a sequence of radio-frequency (RF) images. Kanai et al. (2003) proposed to compute a map of elastic moduli to characterize the carotid artery. Mai and Insana (2002) proposed to monitor deformations in tissues surrounding superficial arteries; a tissue-like gelatin elasticity-flow phantom and in vivo scanning of the normal brachial artery were used to validate the method.

However, most of the conventional methods in elastography only provide the map of the strain distribution in the direction of the ultrasound beam propagation (axial strain, or radial strain in endovascular elastography). Tissue stiffness is conventionally represented with a color code where dark and bright are associated to hard and soft tissues. This can set a potential limitation in non-invasive characterization of vessel walls with an extracorporal ultrasound probe, since the ultrasound beam propagates axially whereas the tissue motion runs radially.

Indeed, it has been shown that motion parameters might be difficult to interpret when tissue motion and the ultrasound beam do not occur in the same orientation. As a consequence of that, the axial and lateral strain parameters are subjected to hardening and softening artifacts, which are to be counteracted to appropriately characterize the vessel wall. These artifacts are also known as mechanical artifacts that can be defined as a wrong association of a strain pattern to hard or soft tissues. Mechanical artifacts originate from a combination of the intrinsic mechanical properties of the underlying tissue, its geometry, and its kinetics.

### B) Non-Invasive Micro-Vascular Elastography (MicroNive)

The following literature review is focusing on the effect of hypertension on the remodeling of the vascular wall. However, the proposed technology is not restricted to this application and concerns imaging of the mechanical structures of small vessels in humans and small animals such as rats and mice. The targeted diseases are not restricted to hypertension and include any pathology affecting the mechanical properties and structures of the vascular wall such as atherosclerosis, for which specific animal models were developed.

Regarding the investigation of the phenotyping in hypertension (HT) with genetically-engineered rat models, it has been shown that the structural and mechanical properties of the arterial tree are altered. Many researchers have investigated such an assertion. For example, Intengan et al. (1998a, 1998b), using DOCA-salt rats, specifically studied the interaction of vasopressin and endothelin 1 (ET-1) in the pathogenesis of the structural vascular alterations. Their results demonstrated that the DOCA-salt model of HT is associated with vascular growth (increased media width, media-lumen ratio, and a growth index of 44%) in the absence of changes in vascular distensibility. On the other hand, many investigations have also been conducted on the role of the proximal arteries in HT. Namely; Cantini et al. (2001) observed an aortic wall stiffness increasing with age in Wistar rats. Tatchum-Talom et al. (2001) demonstrated that the aortic stiffness is decreased in estrogen-deficient rats. Many other researches demonstrated the implication of the proximal arteries in the pathophysiology of HT [Johns et al., (1998); Si et al., (1999); Goud et al., (1998); Zhao et al., (2002)]. However, those experiments were conducted ex vivo and required the sacrifice of the animals. Since the most relevant insights into vascular diseases should come from in situ investigations, there is a need for non-invasive micro-vascular ultrasound elastography (MicroNIVE). The availability of such a mechanical characterization non-invasively could lead to significant new discoveries in functional genomics and pharmacogenetics [Hamet et al., (2002)].

### C) Endovascular Elastography (EVE)

Atherosclerosis, which is a disease of the intima layer of arteries, remains a major cause of mortality in western countries. This pathology is characterized by a focal accumulation of lipids, complex carbohydrates, blood cells, fibrous tissues and calcified deposits, forming a plaque that thickens and hardens the arterial wall. A severe complication of atherosclerosis is thrombosis, a consequence to plaque rupture or fissure, which might lead, according to the event localization, to unstable angina, brain or myocardial infarction, and sudden ischemic death [Falk, (1989); Davies and Thomas (1985); Zaman et al., (2000)]. Plaque rupture is a complicated mechanical process, correlated with plaque morphology, composition, mechanical properties and with the blood pressure and its long term repetitive cycle [Fung, (1993); Falk, (1992)]. Extracting information on the plaque local mechanical properties and on the surrounding tissues may thus reveal relevant features about plaque vulnerability [Fisher et al., (2000); Ohayon et al., (2001)]. Unfortunately no imaging modality, currently in clinical use, allows the access to these properties.

So far, diagnosis and prognosis of atherosclerosis evolution in humans mainly rely on plaque morphology and vessel stenosis degree. This information can accurately be accessed with IntraVascular UltraSound (IVUS) imaging, since this modality provides high resolution cross-sectional images of arteries. Accurate quantitative analysis of the disease is thus easily performed by precise measurements of the lumen area, arterial dimensions and dimensions specific to the plaque. Moreover, IVUS permits the qualitative characterization of plaque components, but roughly, in terms of fatty, fibrous or calcified plaques and with possible misinterpretations. This makes IVUS, alone, insufficient to predict the plaque mechanical behavior. However, elastic properties of vessel walls can be derived from radio-frequency (RF) or alternatively from B-mode IVUS images, by integrating elastographic processing methods. Indeed, endovascular ultrasound elastography (EVE) is an in-development imaging technique that aims to outline elastic properties of vessel walls. Its principle consists of acquiring sequences of cross-sectional vessel ultrasound images, while the vascular tissue is compressed by applying a force from within the lumen. Strain distribution is then estimated by tracking, from the signals, the modifications induced by the stress application. In practice, in EVE, such a stress can be induced by the normal cardiac pulsation or by using a compliant intravascular angioplasty balloon.

Several approaches have been proposed to assess tissue motion in EVE. Whereas one-dimensional (1D) motion estimators are likely more sensitive to pre- and post-motion signal decoherence, two-dimensional (2D) motion estimators are expected to be more reliable. However, the most commonly used motion estimators in EVE applications are 1D correlation-based techniques. This choice is mainly dictated by the ability of such estimators to be implemented; they also may provide real-time tissue motion estimates. In 1D correlation-based tissue motion estimators, the displacement between pre- and post-motion pairs of RF- or B-mode lines is determined using cross-correlation analysis. This technique was used to investigate EVE feasibility on vessel-mimicking phantoms [de Korte et al., (1997)], on excised human femoral and coronary arteries [de Korte et al., (1998); (2000a)], and in vivo on human coronary arteries [de Korte et al., (2000b)].

A different implementation of the 1D cross-correlation technique was proposed by Brusseau et al. (2001) to investigate a post-mortem human excised carotid artery. Brusseau computed an adaptive and iterative estimation of local scaling factors, using the phase information between pre- and post-compression RF signals. The authors suggested that this approach may be less sensitive to decorrelation noise than conventional 1D correlation-based estimators. On the other hand, others also proposed to assess local scaling factors, but in the frequency domain [Thalami et al., (1994)]. They presented some initial in vitro and in vivo results that were obtained with this spectral tissue strain estimator. Envelope B-mode data were used in this last study. However, no further validation of the spectral approach was so far conducted in EVE.

In vivo applications of EVE are subjected to many difficulties. For instance, the position of the catheter in the lumen is generally neither in the center nor parallel to the vessel axis, and the lumen geometry is generally not circular. In such conditions, tissue displacements may be misaligned with the ultrasound beam, introducing substantial decorrelation between the pre- and the post-tissue-compression signals. In addition, although the ultrasound beam propagates close to parallel with the tissue motion in EVE, providing the full strain tensor should improve the characterization of complex heterogeneous tissue structures that may deform unpredictably following the cardiac pulsation of the vessel. The complex heterogeneous nature of plaques may indeed induce 1D decorrelation due to the complex 3D movement of the tissue structures. Regarding that, 1D estimators may not be optimal if such decorrelation is not appropriately compensated for. Ryan and Foster (1997) then proposed to use a 2D correlation-based speckle tracking method to compute vascular elastograms. This approach was experimented on envelope B-mode data from in vitro vessel-mimicking phantoms. No further validation was however conducted by this group.

Another potential difficulty, that is associated with EVE in vivo applications, stems from the eventual cyclic catheter movement in the vessel lumen. Owing to the pulsatile blood flow motion, catheter instability may constitute another source of signal decorrelation between pre- and post-compression signals. To that, Shapo et al. (1996a; 1996b) proposed the use of an angioplasty balloon to stabilize the catheter in the vessel lumen. Tissue motion was assessed using a 2D correlation-based phase sensitive speckle tracking technique. Preliminary results from simulations and from in vitro vessel-mimicking phantom investigations were presented; envelope B-mode data were used.

### OBJECTS OF THE INVENTION

An object of the present invention is therefore to provide an improved method and system for vascular elastography. Another object is to provide a method and system to non-invasively map the elastic properties of vessels.

### SUMMARY OF THE INVENTION

According to a first aspect of the present invention, there is provided a method for vascular elastography comprising:

providing pre-tissue-motion and post-tissue-motion images in digital form of a vessel delimited by a vascular wall; the pre-tissue-motion and post-tissue-motion images being representative of first and second time-delayed configuration of the vessel;

partitioning at least portions of both the pre-tissue-motion and post-tissue-motion images into corresponding data windows;

approximating a trajectory between the pre-tissue-motion and post-tissue-motion images for corresponding data windows; and

using the trajectory for each data window to compute a strain tensor in each data window.

The method can be adapted for non-invasive vascular ultrasound elastography (NIVE) to non-invasively characterize superficial vessels such as carotid, femoral arteries, etc. NIVE is of clinical values for the purpose of diagnosis and follow-up of vascular pathologies.

The method can further be adapted for non-invasive vascular ultrasound micro-elastography (MicroNIVE) for characterizing small superficial vessels in humans and animals. More specifically but not exclusively, MicroNIVE is of value in functional genomics to investigate phenotyping in hypertension with genetically-engineered rat models.

The method for vascular elastography according to the first aspect of the present invention can also be adapted for endovascular ultrasound elastography (EVE) for invasive characterization of vessels using catheter-based techniques. More specifically but not exclusively, EVE is used to investigate coronary diseases in humans.

The method for vascular elastography according to the first aspect of the present invention can also be adapted to other imaging technologies such as, but not exclusively, to magnetic resonance imaging (MRI), optical coherence tomography (OCT) or Doppler-based ultrasound imaging for the non-invasive and invasive characterization of vessels, providing that the imaging techniques can provide the assessment of tissue motion.

According to a second aspect of the present invention, there is provided a system for vascular elastography comprising:

an ultrasound system for acquiring pre-tissue motion and post-tissue motion radio-frequency (RF) images of a vessel; the pre-tissue motion and post-tissue motion images being representative of first and second time-delayed configuration of the vessel;

a controller, coupled to the ultrasound system, i) for receiving the pre-tissue motion and post-tissue motion RF images, ii) for digitizing the pre-tissue motion and post-tissue motion RF images, iii) for partitioning both the pre-tissue motion and post-tissue motion RF images within the vascular wall into corresponding data windows, iv) for approximating a trajectory for each the data windows; and v) for using the trajectory for each the data window to compute a strain tensor in each data window; and

an output device coupled to the controller to output information related to the strain tensor in each data window.

Other objects, advantages and features of the present invention will become more apparent upon reading the following non restrictive description of preferred embodiments thereof, given by way of example only with reference to the accompanying drawings. It is to be noted that the examples presented hereafter were based on the analysis of the ultrasound RF signals. The present invention is not restricted to RF or B-mode signals and may be applied to any new ultrasound modalities providing tissue movements. The RF signals may be seen as the raw data from which all current imaging modalities available on the market were developed.

### BRIEF DESCRIPTION OF THE DRAWINGS

In the appended drawings:

FIG. 1 is a block diagram of a system for vascular elastography according to a first illustrative embodiment of a first aspect of the present invention;

FIGS. 2 and 3 are respectively a flowchart and a block diagram illustrating a method for vascular elastography according to a first illustrative embodiment of a second aspect of the present invention;

FIG. 4 is a schematic view illustrating a two-dimensional partitioning of a region of interest (ROI) within a vascular wall, part of the method illustrated in FIGS. 2 and 3;

FIG. 5 is a block diagram illustrating a method for vascular elastography according to a second illustrative embodiment of the first aspect of the present invention;

FIGS. 6A-6F are theoretical gray-scaled displacement fields and elastograms illustrating motion parameters for a pressurized thick-wall cylindrical blood vessel, embedded in an elastic infinite medium;

FIGS. 7A-7E are theoretical gray-scaled displacement fields and elastograms illustrating radial strain and strain decay for a homogeneous vessel wall;

FIGS. 8A-8C are respectively gray-scaled elastograms (8A-8B) obtained and a graph illustrating the comparison between the radial strain from FIGS. 7 and the Von Mises (VM) parameter;

FIG. 9 is a schematic view of an experimental set-up used to produce mechanical deformation of polyvinyl alcohol cryogel (PVA-C) vessel-mimicking phantoms, and to collect RF ultrasound data incorporating the system from FIG. 1;

FIG. 10 is a schematic view of the vascular flow phantom from the experimental set-up from FIG. 9;

FIGS. 11A-11C are schematic views of the moulds that were used to construct the double-layer PVA-C vessel from FIG. 10;

FIGS. 12A-12C are respectively a B-mode image, a Von Mises (VM or ξ) elastogram obtained using the method from FIG. 2 and the set-up from FIG. 9 and a graph illustrating the average of 5 axial lines chosen in the middle of ξ in the FIG. 12B; FIG. 12A being labeled “Prior Art”;

FIGS. 13A-13B, which are labeled “Prior art”, are respectively B-mode image of a carotid artery acquired from a healthy volunteer, and a manually segmented B-mode image of the vessel wall;

FIGS. 13C-13D, are gray-scaled elastograms computed from data acquired at two different locations of the carotid artery from FIGS. 13A-13B, using the method from FIG. 2;

FIGS. 14A-14B, which are labelled “Prior Art”, are B-mode images acquired over longitudinal sections of the carotid artery of respectively a normotensive and a hypertensive rat;

FIGS. 14C-14H are axial strain “gray-scaled” elastograms of the carotid artery of six different rats, three normotensive (C-E) and three hypertensive (F-H) obtained using the method from FIG. 2;

FIG. 15 is a schematic view illustrating the image acquisition process part of a method for endovascular elastography according to a third illustrative embodiment of the second aspect of the present invention;

FIG. 16 is a schematic view illustrating an “ideal” plaque in a vascular tissue representation;

FIG. 17A-17B are respectively an in vivo intravascular ultrasound cross-sectional image of a coronary plaque and a two-dimensional finite element mesh of the unload real geometry with spatial distribution of the constituents from the plaque from FIG. 17A; FIG. 17A being labeled “Prior Art”;

FIGS. 18A-18D are respectively a theoretical “gray-scaled” elastogram of a radial strain computed for an idealized plaque; a graph illustrating theoretical radial strain distributions taken along the respective lines from FIG. 18A; a radial strain “gray-scaled” elastogram obtained using the endovascular elastography method according to the third illustrative embodiment of the second aspect of the present invention; and a graph illustrating the radial strain distributions taken along the respective lines from FIG. 18C;

FIGS. 19A-19C are respectively a strain-decay-compensated “gray-scaled” elastogram obtained using the endovascular elastography method according to the third illustrative embodiment of the second aspect of the present invention; and one-dimensional vertical and horizontal graphs taken along the respective lines from FIG. 19A;

FIGS. 20A-20C are respectively a theoretical radial strain elastogram of the coronary artery illustrated in FIG. 17A; and one-dimensional vertical and horizontal graphs taken along the respective lines from FIG. 20A;

FIGS. 21A-21C are respectively radial strain “gray-scaled” elastogram computed for the coronary artery illustrated in FIG. 17A using the method for endovascular elastography according to the third illustrative embodiment of the second aspect of the present invention; and one-dimensional vertical and horizontal graphs taken along the respective lines from FIG. 21A;

FIGS. 22A-22C are respectively a strain-decay-compensated “gray-scaled” elastogram of the coronary artery illustrated in FIG. 17A obtained using the endovascular elastography method according to a third illustrative embodiment of the second aspect of the present invention; and one-dimensional vertical and horizontal graphs taken along the respective lines from FIG. 22A;

FIG. 23 is a schematic view of an experimental set-up including a system for endovascular elastography according to a second embodiment of the first aspect of the present invention;

FIGS. 24A-24C, which are labelled “Prior Art”, are respectively a histological section of a post-mortem excised human carotid artery with a very thin plaque; a close-up view of the atherosclerotic region taken from FIG. 24A; and a log-compressed IVUS image of the carotid section; and

FIGS. 25A-25J are “gray-scaled” elastograms computed for consecutive increasing physiologic fluid pressure levels for the carotid artery illustrated in FIGS. 24A-24C using the method for endovascular elastography according to the third illustrative embodiment of the present invention.

### DETAILED DESCRIPTION OF THE INVENTION

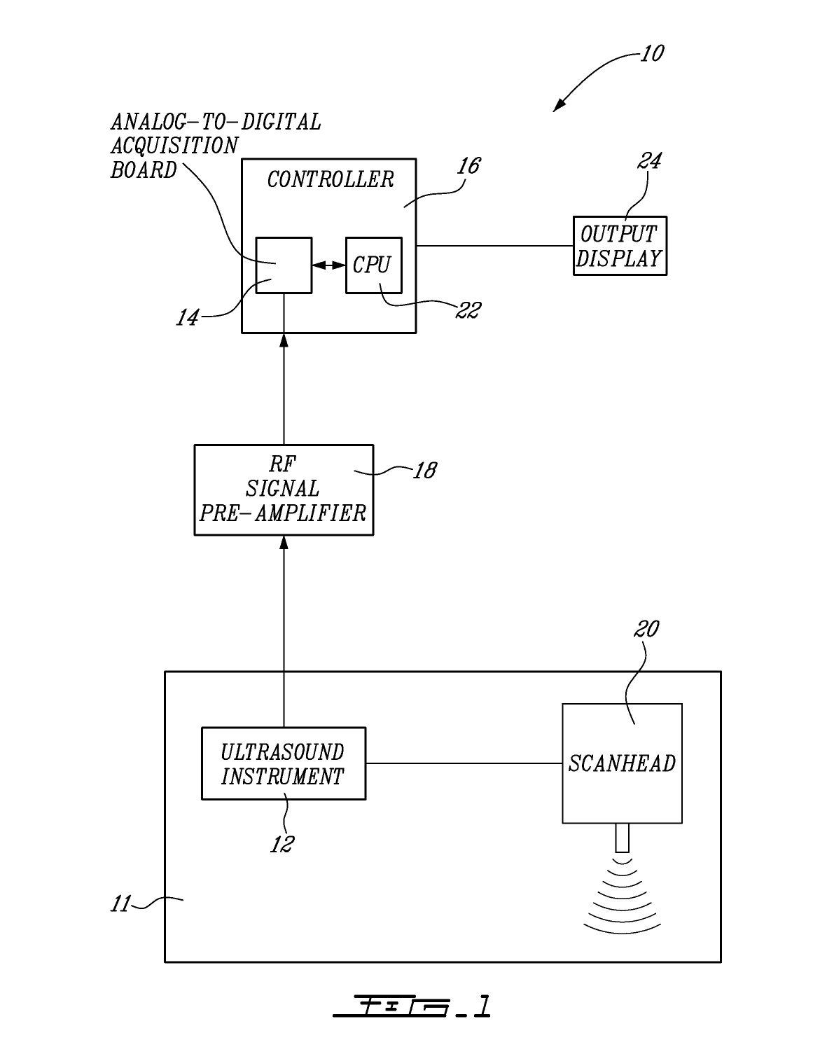

A system 10 for vascular elastography according to a first embodiment of a first aspect of the present invention will now be described with reference to FIG. 1. More specifically, the system 10 allows for non-invasively characterizing arteries. Whereas not restricted to, this system allows predicting risks of vascular tissue rupture due to the presence of atherosclerotic plaques and potentially vascular aneurysms. Since vascular tissue rupture due to atherosclerotic plaques and aneurysms is believed to be well known in the art, it will not be described herein in more detail.

The system 10 comprises an ultrasound system 11 including an ultrasound instrument 12 provided with a scanhead 20 including an ultrasound transducer. The instrument 12 is coupled to an analog-to-digital acquisition board 14 of a controller 16 via a radio-frequency (RF) pre-amplifier 18.

For NIVE, the ultrasound instrument 12 is configured for extracorporal measurement, while for MicroNive, it is in the form of an ultrasound biomicroscope. The ultrasound system 11 is configured with access to RF data so as to allow computing vascular elastograms of vessels. Examples of such ultrasound system 11 are the ES500RP from Ultrasonix for NIVE, and the high-resolution VS-40 or Vevo660 from Visualsonics for MicroNive. An ultrasound system from another type or having other configurations can also be used.

The ultrasound instrument 12 provides an RF output from which the received RF data were transferred to the pre-amplifier 18. An example of pre-amplifier that can be used is the Panametrics, model 5900 PR. Of course, other pre-amplifier can alternatively be used.

The acquisition board 14 allows digitizing the pre-amplified signals from the pre-amplifier 18. An example of acquisition board is the model 8500 CS from Gagescope. Of course, the present invention is not limited to that specific embodiment of acquisition board. A typical sampling frequency is 500 MHz, in 8-bit format.

The controller 16 is in the form of a personal computer including a central processing unit (CPU) 22 which is provided with an output device 24 in the form of a display monitor coupled to the personal computer 16 and input devices such as a keyboard and pointing device also coupled thereto (both not shown). The controller 16 is provided with a memory for storing the scan signals and/or storing information elastogram related information as it will be explained hereinbelow in more detail. The controller 16 may take many other forms including a hand held device, an electronic circuit, a programmed chip, etc.

The controller 16, RF signal pre-amplifier 18 and/or ultrasound system 11 may be part of a single vascular elastography device.

The controller 16 is configured and programmed so as to implement a method for vascular elastography as it will be described furthering.

In operation of the system 10, the ultrasound transducer of the ultrasound system 11 is applied on the skin over the region of interest, and the arterial tissue is dilated by the cardiac pulsation or any other arterial tissue dilatation means.

The elastograms, the equivalent elasticity images, are computed from the assessment of the vascular tissue motion as it will be explained hereinbelow in more detail. For that purpose, longitudinal or/and cross-sectional RF data are measured. For longitudinal investigations, because they are more convenient, axial deformation parameters may be sufficient to characterize the vessel wall. For cross-section data, the full strain tensor is used to compute the Von Mises parameter, because motion parameters might be difficult to interpret since tissue motion occurs radially within the vessel wall while the ultrasound beam propagates axially. As a consequence of that, the elastograms are subjected to hardening and softening artifacts, which are to be counteracted.

As it will now be described herein in more detail with reference to a method 100 for vascular elastography, according to a first illustrative embodiment of a second aspect of the present invention, the Von Mises (VM) coefficient is computed in order to circumvent such mechanical artifacts and to appropriately characterize the vessel wall. More specifically, a Lagrangian speckle model estimator (LSME) is used to model the vascular motion which provides the full strain tensor for computing the VM coefficient.

The method 100, which is illustrated in FIGS. 2-3, comprises the following step:

102—Providing a sequence of radio-frequency (RF) images, including at least one pre-tissue-motion and at least one post-tissue-motion image, of a vessel delimited by a vascular wall;

104—Partitioning both RF images within the vascular wall into corresponding data windows;

106—Approximating a trajectory between the pre- and post-tissue-motion for corresponding data windows; and

108—Using the data window trajectories to compute a strain tensor in each data window.

Each of these steps will now be described in more detail.

In step 102, a time-sequence of one-dimensional (1D) I(x(t)), two-dimensional (2D) I(x(t), y(t)) or three-dimensional (3D) RF images I(x(t), y(t), z(t)) is provided, among which two images are selected for steps 104-108. The first image I(x(t₀), y(t₀), z(t₀)) will be referred to as the pre-tissue-motion image and the second image I(x(t₀+Δt), y(t₀+Δt), z(t₀+Δt)) will be referred to as the post-tissue-motion image. Images obtained through other imaging modalities than ultrasound can also be used.

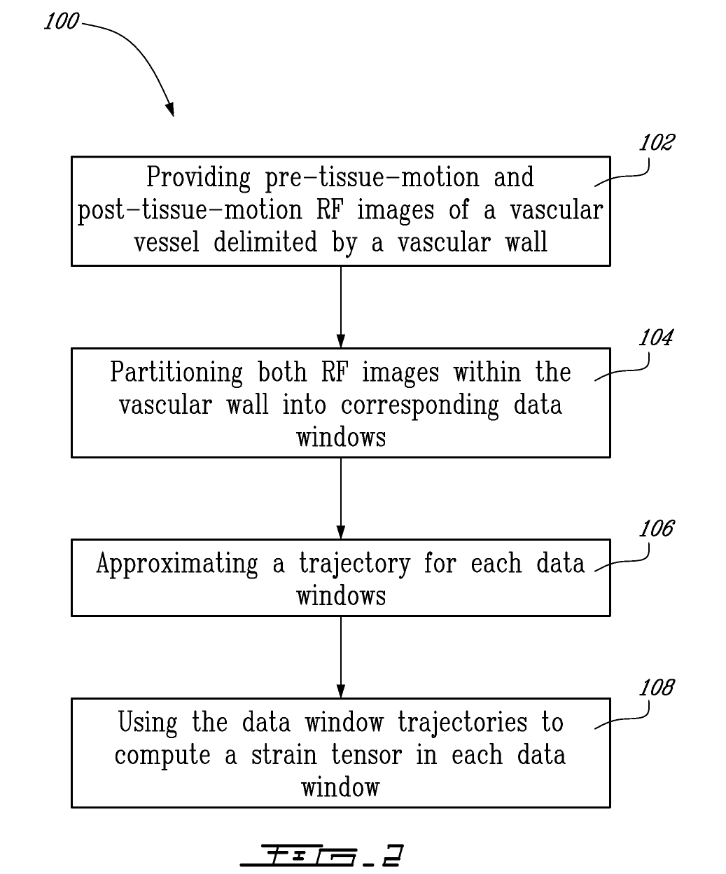

In step 104, both selected RF images are partitioned within the vascular wall into corresponding data windows Wᵢⱼ. FIG. 4 illustrates an example of two-dimension partitioning of the region of interest (ROI) into Wₘₙ windows. Of course, the partitioning of the ROI can be in 1D or extended in three-dimension.

The vascular tissue and boundary conditions are generally heterogeneous. The vessel wall is thus expected to deform non-uniformly. As illustrated in FIG. 4, to assess such tissue motion, the method 100 includes subdividing the ROI within the vascular wall into several partitions Wᵢⱼ, for which motion can be assumed as affine.

In step 106, a trajectory is approximated for each data windows by the zero-order and first-order terms of a Taylor-series expansion. For a three-dimensional tissue, assuming that the origin is set at (0,0,0), this can be expressed as:

$$[ \begin{matrix}x \\ y \\ z\end{matrix} ] = {\underbrace{[ \begin{matrix}x ( 0 , 0 , 0 , t ) \\ y ( 0 , 0 , 0 , t ) \\ z ( 0 , 0 , 0 , t )\end{matrix} ]}}_{Tr} + {\underbrace{{[ \begin{matrix}\frac{\partial x}{\partial {x}_{0}} & \frac{\partial x}{\partial {y}_{0}} & \frac{\partial x}{\partial {z}_{0}} \\ \frac{\partial y}{\partial {x}_{0}} & \frac{\partial y}{\partial {y}_{0}} & \frac{\partial y}{\partial {z}_{0}} \\ \frac{\partial z}{\partial {x}_{0}} & \frac{\partial z}{\partial {y}_{0}} & \frac{\partial z}{\partial {z}_{0}}\end{matrix} ]}_{( 0 , 0 , 0 , t )}}}_{LT} [ \begin{matrix}{x}_{0} \\ {y}_{0} \\ {z}_{0}\end{matrix} ] . \qquad ( 1 )$$

Equation 1 defines an affine transformation, i.e. it is the result of a translation (vector [Tr]) and of a linear geometrical transformation of coordinates (matrix [LT]). Equation 1 can also be seen as representing trajectories that describe a tissue motion in a region of constant strain. Strain is usually defined in terms of the gradient of a displacement field; hence, as (x, y, z) represents the new position of a point (x₀, y₀, z₀), the (u, v, w) displacement vector in the Cartesian coordinate system is given by:

$$[ \begin{matrix}u \\ v \\ w\end{matrix} ] = [ \begin{matrix}x - {x}_{0} \\ y - {y}_{0} \\ z - {z}_{0}\end{matrix} ] = {\underbrace{[ \begin{matrix}x ( 0 , 0 , 0 , t ) \\ y ( 0 , 0 , 0 , t ) \\ z ( 0 , 0 , 0 , t )\end{matrix} ]}}_{Tr} + Δ [ \begin{matrix}{x}_{0} \\ {y}_{0} \\ {z}_{0}\end{matrix} ] , with \text{:} \\ Δ = {\underbrace{{[ \begin{matrix}\frac{\partial x}{\partial {x}_{0}} - 1 & \frac{\partial x}{\partial {y}_{0}} & \frac{\partial x}{\partial {z}_{0}} \\ \frac{\partial y}{\partial {x}_{0}} & \frac{\partial y}{\partial {y}_{0}} - 1 & \frac{\partial y}{\partial {z}_{0}} \\ \frac{\partial z}{\partial {x}_{0}} & \frac{\partial z}{\partial {y}_{0}} & \frac{\partial z}{\partial {z}_{0}} - 1\end{matrix} ]}_{( 0 , 0 , 0 , t )}}}_{LT - 1} \qquad ( 2 )$$

In this Equation, [I] is the 3D identity matrix. The εᵢⱼ, which are the components of the 3D-strain tensor ε, can then be defined in terms of Δ (the deformation matrix) as:

$${ɛ}_{ij} ( t ) = \frac{1}{2} [ {Δ}_{ij} ( t ) + {Δ}_{ji} ( t ) ] \qquad ( 3 )$$

Given Equation 3, the Von Mises (VM) coefficient can be computed [Mase, 1970]. VM is independent of the coordinate system and is mathematically expressed as:

$$ξ = {\{ \begin{matrix}\frac{2}{9} [ {( {ɛ}_{xx} - {ɛ}_{yy} )}^{2} + {( {ɛ}_{yy} - {ɛ}_{zz} )}^{2} + \\ {( {ɛ}_{zz} - {ɛ}_{xx} )}^{2} + 6 ( {ɛ}_{xy}^{2} + {ɛ}_{yz}^{2} + {ɛ}_{xz}^{2} ) ]\end{matrix} \}}^{1 / 2} \qquad ( 4 )$$

For each partition window within the vascular wall, ξ can be related to the elastic modulus (E) through the following relationship:

$$E = \frac{σ}{ξ} . \qquad ( 5 )$$

where σ depends on the pressure gradient that results from the blood flow pulsation or from the pressurization of the vessel by an external means.

In step 108, the deformation matrix (Δ) is computed in each data window using the data window trajectories.

More specifically, a non-linear minimization is performed for each by computing the [LT] that allows the best match between each Wᵢⱼ of the pre-tissue motion image and its counterpart or corresponding window in the post-tissue motion image. In other words, the method 100 yields the deformation matrix (Δ) and the strain tensor (ε) through Equations 1, 2 and 3. The map of the distribution of each component of the deformation matrix (Δ) provides a unique elastogram; the components of ε can also be combined to provide a composite elastogram as it is the case for the VM coefficient (Equation 4). In Cartesian coordinates, ε₁₁ (=εₓₓ), ε₂₂ (=ε_{yy}) and ε₃₃ (=ε_{zz}) are referred to as lateral, axial and transverse elastograms, respectively. The ε₁₂ (=ε₂₁ =ε_{xy} =ε_{yx}), ε₁₃ (=ε₃₁ =ε_{xz} =ε_{zx}) and ε₂₃ (=ε₃₂ =ε_{yz} =ε_{zy}) are the shear elastograms available with this technology. As it is believed to be well known in the art, elastograms are usually presented as color-code images where dark and bright regions are conventionally associated to hard and soft tissues.

Step 108 that is referred to herein as the Lagrangian Speckle Model estimator (LSME), can be mathematically expressed as:

$$\begin{matrix}MIN \\ {Ψ}_{ij}\end{matrix} {‖ I ( x ( {t}_{0} ) , y ( {t}_{0} ) , z ( {t}_{0} ) ) - {I}_{Lag} ( \begin{matrix}x ( {t}_{0} + Δ t ) , y ( {t}_{0} + Δ t ) , \\ z ( {t}_{0} + Δ t )\end{matrix} ) ‖}^{2} \qquad ( 6 )$$

with ψᵢⱼ=[Tr;LT(:)] for a given Wᵢⱼ using the notation for augmented vector (;) and matrix vectorisation (:). Hence, ωᵢⱼ is a 12×1 vector built from the 3×1 Tr vector and the 9×1 vectorisation of LT. I_{Lag} (x(t₀+Δt), y(t₀+Δt), z(t₀+Δt)) is the Lagrangian speckle image (LSI); it is defined as the post-tissue motion RF image I(x(t₀+Δt), y(t₀+Δt), z(t₀+Δt)) that was numerically compensated for tissue motion, as to achieve the best match with I(x(t₀), y(t₀), z(t₀)) [Maurice and Bertrand, 1999]. The appellation “Lagrangian” refers to the Lagrangian description of motion. The minimum of Equation 6 is obtained by using the appropriate [ψᵢⱼ] that best matches I₀=I(x(t₀),y(t₀),z(t₀)) and I₁=I(x(t₀+Δt),y(t₀+Δt),z(t₀+Δt)). ψ₀ is the initial guess to start the iterative process. The regularized nonlinear Levenberg-Marquardt (L&M) minimization algorithm [Levenberg, 1963; Marquardt, 1944] is used in solving Equation 6. Of course, other minimization algorithms can also be used.

The method 100 allows computing the full 3D-strain tensor (Equation 3). Whereas the divergence parameters (εₓₓ, ε_{yy} and ε_{zz}) provide information about tissue stiffness, the shear parameters (ε_{xy}, ε_{xz} and ε_{yz}) can provide useful insights on the heterogeneous nature of the vessel wall.

A method 200 for vascular elastography according to a second illustrative embodiment of the present invention will now be described with reference to FIG. 5. Since the method 200 is very similar to method 100, and for concision purposes, only the differences between the two methods will be described furthering.

The optical flow-based method 200 is based on the assumption that speckle behaves as a material property.

As illustrated in FIG. 5, the cross-correlation analysis provides 3D displacement fields and a correlation map between I₀ and I₁. Tissue motion parameters (Δ, tᵢⱼ) are computed for each Wᵢⱼ using Iₒ and I_{Lag}.

The material derivative of a function I(x(t), y(t), z(t)) is given as:

$$\frac{d I}{d t} = {I}_{x} \frac{d x}{d t} + {I}_{y} \frac{d y}{d t} + {I}_{z} \frac{d z}{d t} + {I}_{t} , \qquad ( 7 )$$

with Iₓ, I_{y}, I_{z}, and Iₜ being the partial derivatives of I(x(t), y(t), z(t)) with respect to x, y, z and t, respectively. Here, Iₜ is the time rate of change of I(x(t), y(t), z(t)) in the observer coordinate system,

$$( \frac{d x}{d t} , \frac{d y}{d t} , \frac{d z}{d t} )$$

is the velocity vector of a “material point” located at (x,y,z), and

$$\frac{d I}{d t}$$

is the intrinsic rate of change of the material point. For a time-sequence of two images at interval dt, Equation 7 can be rewritten as:

dI=Iₓdx+I_{y}dy+I_{z}dz+(I(x(t+dt),y(t+dt),z(t+dt))−I(x(t),y(t),z(t)) (8)

where (dx, dy, dz) represents the displacement vector of the “material point” located at (x,y,z) in the time interval dt.

Furthermore, assuming speckle being a material property that is preserved with motion (dI(x(t),y(t),z(t))=0), and assuming I(x(t+dt),y(t+dt),z(t+dt)) (equivalently, I_{Lag}(x(t+dt),y(t+dt),z(t+dt))) being a very close approximation of I(x(t),y(t),z(t)), one obtains:

Iₓdx+I_{y}dy+I_{z}dz=−(I_{Lag}(x(t+dt),y(t+dt),z(t+dt))−I(x(t),y(t),z(t))) (9)

Now, with respect to Equations 2 and 3, Equation 9 can be rewritten as:

$$[ \begin{matrix}( {t}_{x} + {Δ}_{xx} x + {Δ}_{xy} y + {Δ}_{xx} z ) \\ ( {t}_{y} + {Δ}_{yx} x + {Δ}_{yy} y + {Δ}_{yz} z ) \\ ( {t}_{z} + {Δ}_{zx} x + {Δ}_{zy} y + {Δ}_{zz} z )\end{matrix} ] [ \begin{matrix}{I}_{x} \\ {I}_{y} \\ {I}_{z}\end{matrix} ] = - {\tilde{I}}_{t} \qquad 10 )$$

For the purpose of clarity, the following simplifications are made in Equation 10: tₓ=x(0,0,0,t); t_{y}=y(0,0,0,t); t_{z}=z(0,0,0,t); and Ĩₜ=(I_{Lag}(x(t+dt),y(t+dt),z(t+dt))−I(x(t),y(t),z(t))).

Finally, if the ROI has a size of p×q pixels, the discrete form of Equation 10 can be written as:

$$[ \begin{matrix}{I}_{{x}_{1}} {x}_{1} & {I}_{{x}_{1}} {y}_{1} & {I}_{{x}_{1}} {z}_{1} & {I}_{{x}_{1}} & ⋯ & {I}_{{z}_{1}} {x}_{1} & {I}_{{z}_{1}} {y}_{1} & {I}_{{z}_{1}} {z}_{1} & {I}_{{z}_{1}} \\ {I}_{{x}_{2}} {x}_{2} & {I}_{{x}_{2}} {y}_{2} & {I}_{{x}_{2}} {z}_{2} & {I}_{{x}_{2}} & ⋯ & {I}_{{z}_{2}} {x}_{2} & {I}_{{z}_{2}} {y}_{2} & {I}_{{z}_{2}} {z}_{2} & {I}_{{z}_{2}} \\ ⋮ & ⋮ & ⋮ & ⋮ & & ⋮ & ⋮ & ⋮ & ⋮ \\ & & & & & & & & \\ & & & & & & & & \\ & & & & & & & & \\ ⋮ & ⋮ & ⋮ & ⋮ & ⋯ & ⋮ & ⋮ & ⋮ & ⋮ \\ & & & & & & & & \\ & & & & & & & & \\ & & & & & & & & \\ ⋮ & ⋮ & ⋮ & ⋮ & ⋯ & ⋮ & ⋮ & ⋮ & ⋮ \\ {I}_{{x}_{p \times q}} {x}_{p \times q} & {I}_{{x}_{p \times q}} {y}_{p \times q} & {I}_{{x}_{p \times q}} {z}_{p \times q} & {I}_{{x}_{p \times q}} & ⋯ & {I}_{{z}_{p \times q}} {x}_{p \times q} & {I}_{{z}_{p \times q}} {y}_{p \times q} & {I}_{{z}_{p \times q}} {z}_{p \times q} & {I}_{{z}_{p \times q}}\end{matrix} ] [ \begin{matrix}{Δ}_{xx} \\ {Δ}_{xy} \\ {Δ}_{xz} \\ {t}_{x} \\ {Δ}_{yx} \\ {Δ}_{yy} \\ {Δ}_{yz} \\ {t}_{y} \\ {Δ}_{zx} \\ {Δ}_{zy} \\ {Δ}_{zz} \\ {t}_{z}\end{matrix} ] = [ \begin{matrix}{\tilde{I}}_{{t}_{1}} \\ {\tilde{I}}_{{t}_{2}} \\ ⋮ \\ \\ \\ \\ ⋮ \\ \\ \\ \\ ⋮ \\ {\tilde{I}}_{{t}_{p \times q}}\end{matrix} ] \qquad ( 11 )$$

Solving Equation 11 yields Δ, tₓ, t_{y} and t_{z} for each pixel of the ROI. One hypothesis supporting Equation 11 is that (dI(x(t),y(t),z(t))=0), i.e. that the intensity of each pixel remained the same following tissue motion. In practice, however, because of signal decorrelation, that is rarely the case. Equation 11 then provides a solution for the minimization problem given in Equation 6. The main advantage of method 200 over the method 100 is relative to the processing time. Indeed, the computation time is improved by a factor close to 25.

As illustrated in FIG. 5, it is to be noted that this implementation of the LSME uses cross-correlation analysis to compute motion compensation as to provide I_{Lag} (x(t+dt),y(t+dt),z(t+dt)). Interestingly, a total of 16 parameters can be assessed in 3D (9 parameters in 2D). Nevertheless, the strain parameters are so far the most convenient for the purpose of characterizing soft tissue mechanical properties.

### Non-Invasive Vascular Elastoqraphy (NIVE and MicroNive)

### Tissue Motion Analysis for Cross-Section Data

Superficial arteries such as the carotid and femoral are easily accessible and can be imaged longitudinally. This can be seen as the most convenient application of the method 100 in non-invasive vascular elastography (NIVE), since tissue motion can be expected to run close to parallel to the ultrasound beam. In this context, the axial components of the deformation matrix may be sufficient to characterize the vessel wall. Whereas tissue motion analysis for longitudinal data will be presented with reference to a further illustrative embodiment of the present method, we here emphasize on cross-sectional data.

For a continuum, motion can be described in a Lagrangian coordinate system or in an Eulerian coordinate system [Le Mehaute, 1976; Hinze, 1975]. In the literature, the Eulerian coordinate system is sometimes referred to as the observer's coordinate system, whereas the Lagrangian coordinate system is known as the material coordinate system. The material coordinates allow expressing each portion of the continuum as a function of time and position. Whereas the mathematical formulation (Equations 1-4) is in 3D, the illustration is here presented in 2D for the purpose of simplification. The difference between these two coordinate systems is illustrated in FIG. 4, where the (x,y) constitutes the observer's coordinates and the (r,φ) defines the material coordinates.

With most imaging systems, such as ultrasonography, the observer's and the material coordinate systems are generally the same; hence, most tissue motion estimators use, by definition, the observer's coordinate system. However, the material coordinates can be presented as a suitable way to describe speckle dynamics [Maurice and Bertrand, (1999)].

As illustrated in FIG. 4, the observer's coordinate system is the Cartesian (x,y)-plane. This system is different from the motion coordinate system that is in the radial (r,φ)-plane. In such a situation, the parameters of an estimator are expected to be very difficult to interpret. One of the challenges of non-invasive vascular elastography, regarding cross-section data, concerns the interpretation of the estimated motion parameters to characterize the vascular tissue.

### Simulated and Experimental In Vitro Results of NIVE and MicroNive

As it will now be described in more detail with reference to a pathology free simulation, the VM coefficient can be used as a tissue characterization parameter to better interpret the displayed images.

### Motion Analysis for a Homogenous Tissue

A pathology-free application simulation will now be considered, that is the case of a circular, axis-symmetric and homogeneous vessel section. To take into account the constraints induced by the environmental tissues and organs, it is hypothesized that the vessel section is embedded in an infinite medium of higher Young's modulus. A pressurized thick-wall cylindrical blood vessel of inner and outer radii Rᵢ and Rₒ respectively, embedded in an elastic coaxial cylindrical medium of radius Rₑ, is considered. It is assumed that the plane strain condition for the vessel wall applies and also that the two media are incompressible and isotropic. Referring to Equation 2, the displacement gradient components (Equation 2) are given by:

$$\begin{matrix}Δ ( x , y ) = [ \begin{matrix}{K}_{∞} \frac{{y}^{2} - {x}^{2}}{{( {x}^{2} + {y}^{2} )}^{2}} & - 2 {K}_{∞} \frac{xy}{{( {x}^{2} + {y}^{2} )}^{2}} \\ - 2 {K}_{∞} \frac{xy}{{( {x}^{2} + {y}^{2} )}^{2}} & {K}_{∞} \frac{{x}^{2} - {y}^{2}}{{( {x}^{2} + {y}^{2} )}^{2}}\end{matrix} ] \\ with \text{:} & ( 12 ) \\ {K}_{∞} = \frac{3}{2} {{P}_{b} [ {E}^{( 1 )} ( \frac{1}{{R}_{i}^{2}} - \frac{1}{{R}_{o}^{2}} ) + \frac{{E}^{( 2 )}}{{R}_{o}^{2}} ]}^{- 1} & ( 13 )\end{matrix}$$

where the subscripts (1) and (2) describe respectively the vessel wall and the external medium; Pb defines the blood pressure and E is the Young's modulus.

Equations 12 and 13 were implemented to simulate the dynamics of a homogeneous vessel section subjected to an intraluminal pressure. Close to 6% intraluminal dilation was induced regarding the constitutive model presented here. It is to be noted that 6% intraluminal dilation is equivalent to 3% compression of the intraluminal wall. The physical vessel dimensions were 7-mm outer diameter and 4-mm inner diameter as to approximate the physiological case of a femoral artery.

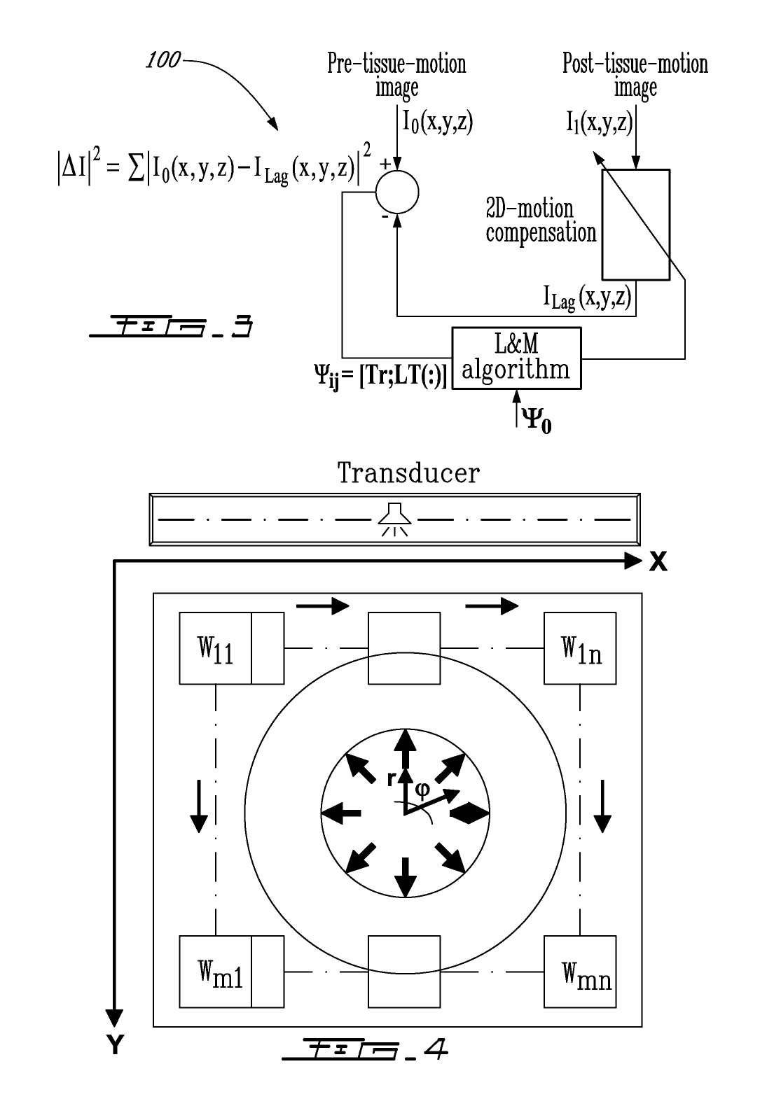

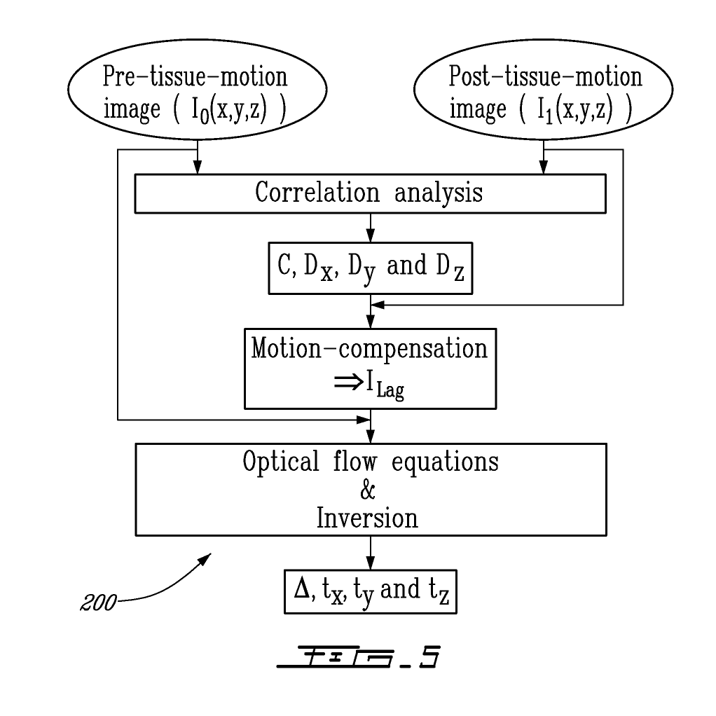

FIGS. 6A and 6B present respectively the lateral and axial displacement fields; they include gray-scale “colorbar” expressing the displacement in μm (10⁻⁶ m). Maximum motion occurred at the lumen interface. FIGS. 6C to 6F present the Δᵢⱼ components of Equation 12, which are respectively the lateral strain, the lateral shear, the axial shear and the axial strain; they include gray-scale “colorbar” expressing the strain in percentage.

Δ_{yy} is expected to be less or equal to zero (≦0) since, in conventional elastography, an external force is applied and induces tissue compression. Traditionally, smaller strain amplitude values are associated with harder regions and are printed dark; equivalently, higher strain amplitude values are associated with softer regions and are printed bright. However according to the method 100, as it can be observed in FIG. 6F, dilation can also be detected (Δ_{yy}≧0) in the elastogram. In an elastographic sense, the dilation regions can be misinterpreted as soft tissue. Indeed, in FIG. 6F, two harder zones (Δ_{yy}≦0) likely seem to be identified at 12 and 6 o'clock. Because, for the conditions simulated, the vessel wall is homogeneous; such a phenomenon is referred to as hardening artifact [Ophir et al., (1999)]. Inversely, two softer zones (Δ_{yy}≧0) at 3 and 9 o'clock seem also to be identified; they are referred to as softening artifacts. Such motion artifacts stem from the fact that motion occurs radially and is observed in Cartesian coordinates.

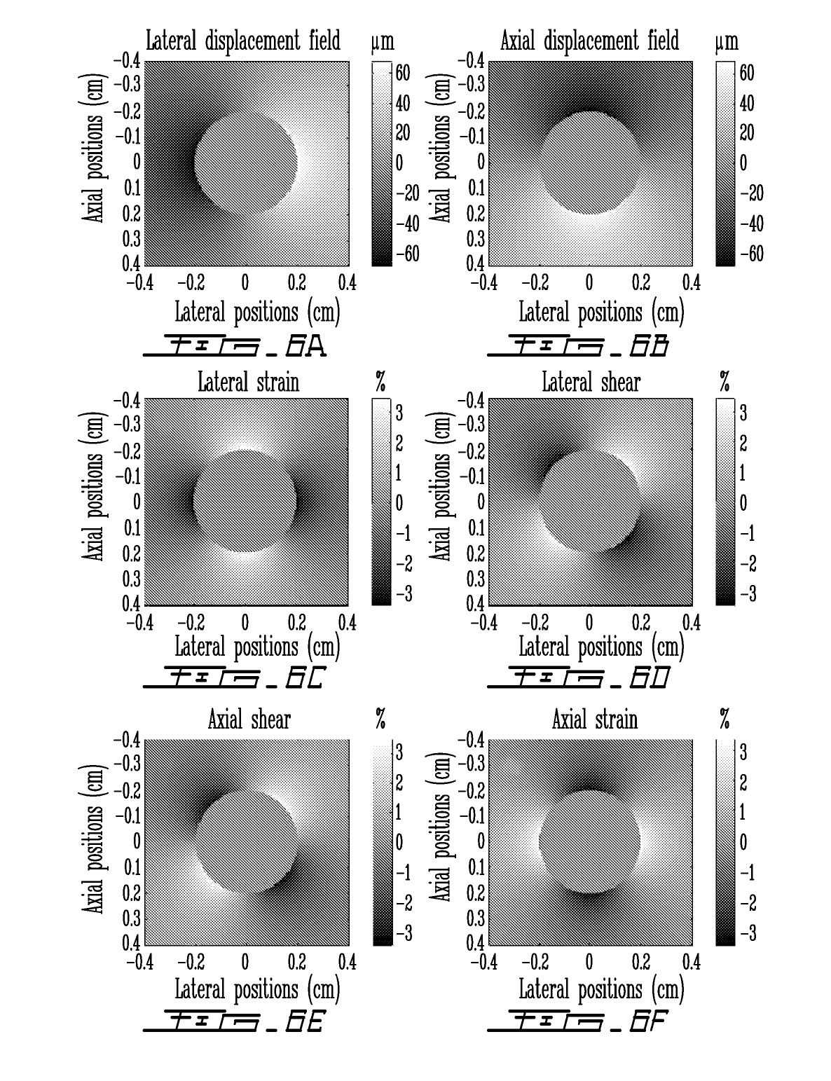

The motion parameters in their natural polar coordinate system or material coordinate system will now be considered. In FIG. 7A, the radial displacement field is computed from the lateral and axial displacement fields (FIGS. 6A and 6B respectively). The radial displacement field is also presented in a polar (r,φ) coordinate system (FIG. 7B). The gradient of the latter displacement field thus provides the radial strain (FIG. 7C). FIG. 7D shows a plot of the radial strain at φ=π. One can observe the monotonic profile of this plot, being maximal at the lumen and minimal at the outer side of the vessel. Such a phenomenon is a consequence of the geometry and is known in the literature as the strain decay [Ryan and Foster, (1997)]. Finally in FIG. 7E, the radial strain is reported back in the (x,y) coordinate system. Regardless of the strain decay phenomenon, one can observe that no specific hard or soft region is identified. FIG. 7E thus illustrates a strain profile that adequately represents a homogenous vessel wall behavior.

Hopefully, elastograms such as the one shown in FIG. 7E allows to appropriately characterizing the vessel wall. However, in NIVE or MicroNive, motion is studied in the transducer coordinate system; that is the (x,y)-Cartesian coordinates. Accordingly, elastograms are expected to be as artifactual as the one in FIG. 6F. The VM coefficient (Equation 4) is then used to characterize the vessel wall [Mase, 1970].

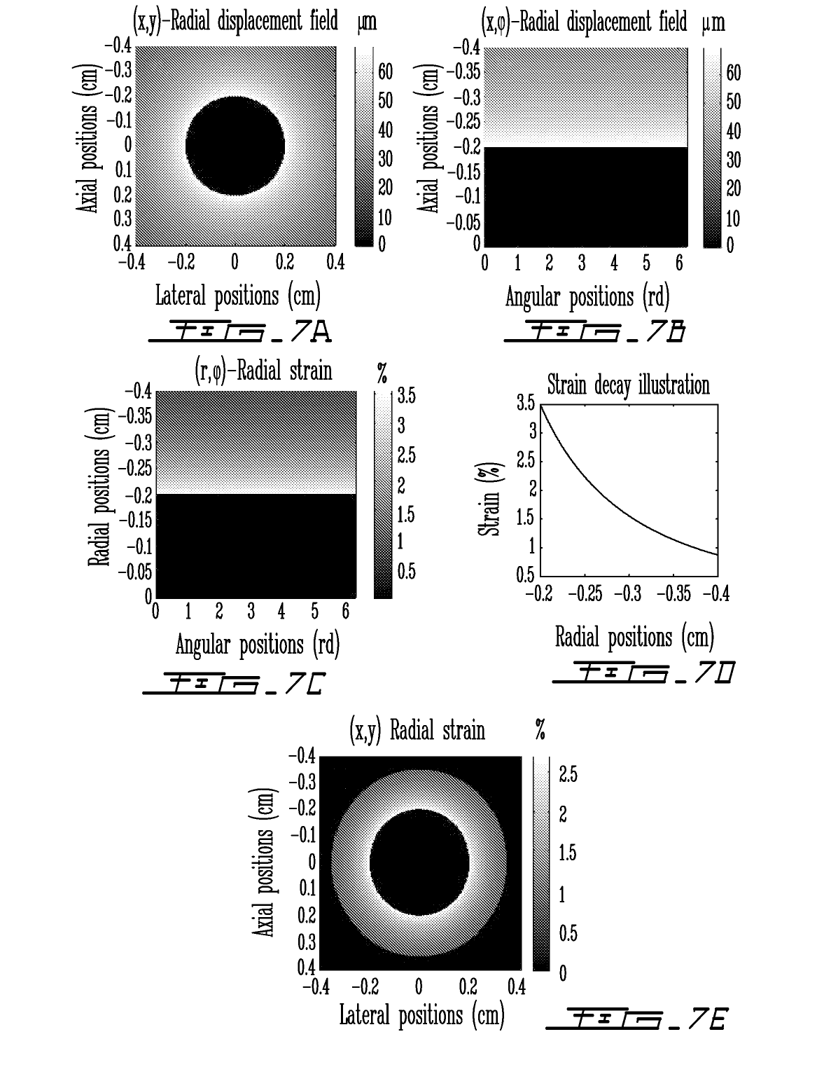

A comparison between the radial strain and the Von Mises parameter (ξ) is shown in FIGS. 8A-8B for a homogeneous vessel wall. Qualitatively, both parameters are equivalent. FIG. 8C shows the plots for the radial strain (—) and (---) at x=0. Those graphs show that the VM coefficient likely improves the contrast between higher and lower strains while the profile remains the same. Moreover, regardless of the strain decay, ξ (as well as the radial strain) is interestingly free of hardening or softening artifacts and is thus suitable to non-invasively characterize the vessel wall. To corroborate such an assumption, a more complex geometry, that is a heterogeneous vessel wall, has been investigated in vitro and the results are presented in the following section

MicroNIVE experiments have been conducted on vessel-mimicking phantoms with a lumen diameter of 1.5 mm and a wall thickness of 2 mm. The phantoms were made of polyvinyl alcohol cryogel (PVA-C).

An experimental set-up 26, used to produce mechanical deformation of polyvinyl alcohol cryogel (PVA-C) vessel-mimicking phantoms, and including the system 10 to collect RF ultrasound data that can be used in computing vascular elastograms according to the method from 100 will now be described with reference to FIG. 9.

A mixture of water-glycerol was circulated in a flow phantom 30. The height difference between the top and bottom reservoirs 28 and 36 allowed adjustment of the gravity-driven flow rate and static pressure within the lumen of the phantom 30. A peristaltic pump 38 was used to circulate the fluid from the bottom to the top reservoirs 36 and 28. The flow rate was measured with an electromagnetic flowmeter 32, which was a Cliniflow II, model FM 701D from Carolina Medical, and the pressure was monitored by a MDE Escort instrument 34, which was a model E102 from Medical Data Electronics. As illustrated in FIG. 9, the flow phantom 30 was not directly connected to the tubing of the top reservoir 28 to facilitate the small incremental pressure step adjustments necessary to obtain correlated deformation of the RF signals within the PVA-C vessel wall.

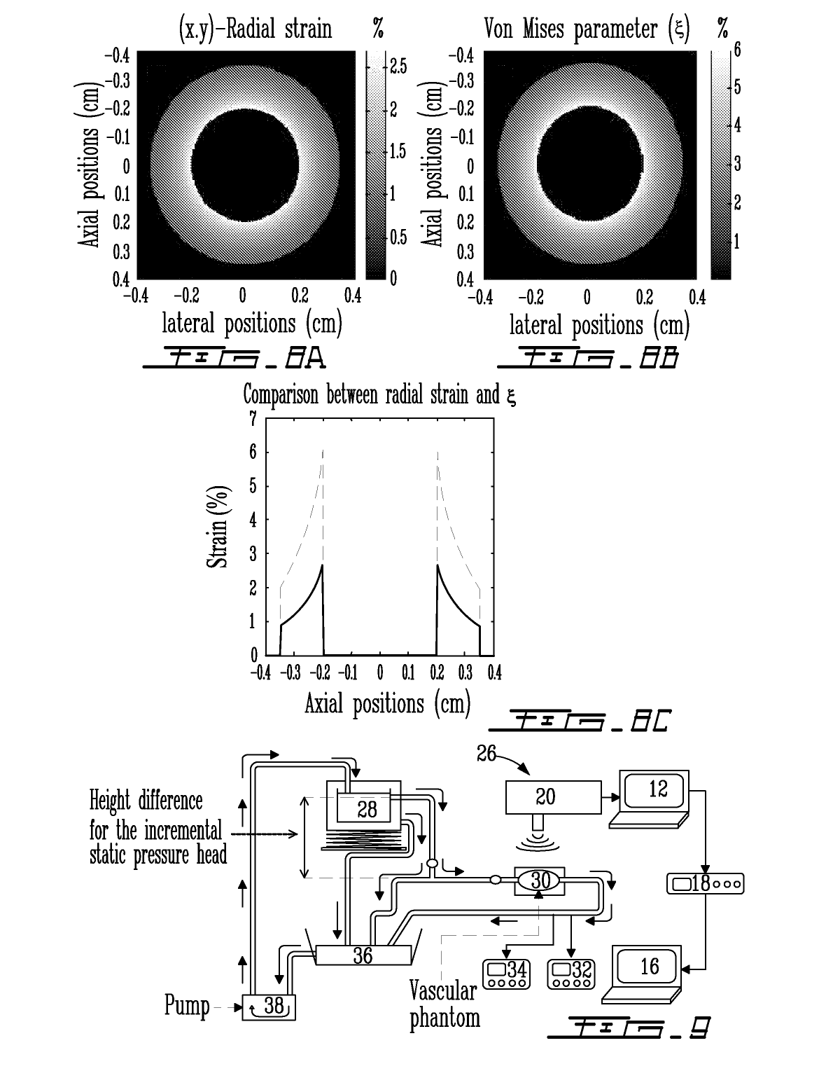

As can be seen in FIG. 10, the polyvinyl alcohol cryogel PVA-C vessel 39 of the flow phantom 30 was positioned between two watertight connectors 40, in a Plexiglas box 42 filled with degassed water 44 at room temperature. Rubber o-rings were used to tight the PVA-C vessel 39 onto Plexiglas tubes 46 at both extremities.

Based on previous works by Chu and Rutt (1997), the tissue-mimicking vessel 39 was made of PVA-C. This biogel solidifies and acquires its mechanical rigidity by increasing the number of freeze/thaw cycles. Indeed, the number of freeze/thaw cycles modifies the structure of the material by increasing the reticulation of fibers. It has been shown that the elastic and acoustic properties of PVA-C are in the range of values found for soft biological tissues [Chu and Rutt, 1997]. More specifically, it has been demonstrated that the stress-strain relationship can be very close to that of a pig aorta.

The vessel-mimicking phantoms 30 approximately had a 1.5-mm lumen diameter, 2-mm wall thickness, and 52-mm length. A 1.5% by weight of Sigmacell (type 20, #S-3504, from Sigma-Aldrich) was added to the PVA-C to provide acoustical scatterers.

Results for one double-layer vessel will now be presented. Each layer had a thickness close to 1 mm, and the inner layer was made softer than the outer one. The numbers of freeze-thaw cycles were set at 2 and 4 for the inner and the outer portions of the wall, respectively. Each freeze-thaw cycle took 24 hours and the temperature was incrementally varied from −20 C to 20 C, by using a specifically designed electronic controller (Watlow, model 981) and a freezer equipped with heated elements such as Supra Scientifique's model YF-204017.

FIGS. 11A-11C show a schematic representation of the moulds that were used to construct the double-layer vessel-mimicking phantoms 39, the simulated vessel having a 1.5 mm lumen diameter, a 2 mm wall thickness (roughly 1 mm for each layer), and a 52 mm length. At a first instance, PVA-C was poured between the first and second templates; that underwent (nₒ−nᵢ) freeze/thaw cycles to provide the external layer. For the purpose of clarity, nᵢ and nₒ as the numbers of cycles for the inner and the outer layers, respectively. Secondly, fresh PVA-C was poured between the second and third templates, while maintaining the first template in place; that underwent nᵢ freeze/thaw cycles to provide a complete double-layer vessel-mimicking phantom.

Returning to FIG. 9, the ultrasound biomicroscope 12 (Visualsonics, model VS-40) provides an RF output from which the received RF data were transferred to a pre-amplifier 18 (Panametrics, model 5900 PR). After amplification, the signals were digitized with an acquisition board 14 (Gagescope, model 8500 CS) installed in a personal computer 12. The sampling frequency was 500 MHz, in 8-bit format.

The double layer vessel-mimicking phantoms 30 measured 5.5 mm in outer diameter, whereas the RF images extended to 8 mm×8 mm. Measurement-windows (partitions or ROI) were of 272 μm×312 μm (200 samples×20 RF lines), with 85% axial and lateral overlaps. The estimated motion parameters were post-processed using a 5×5 kernel Gaussian-filter. The pressure pre-load was 10 mmHg, and the pressure gradient was 5 mmHg between subsequent images.

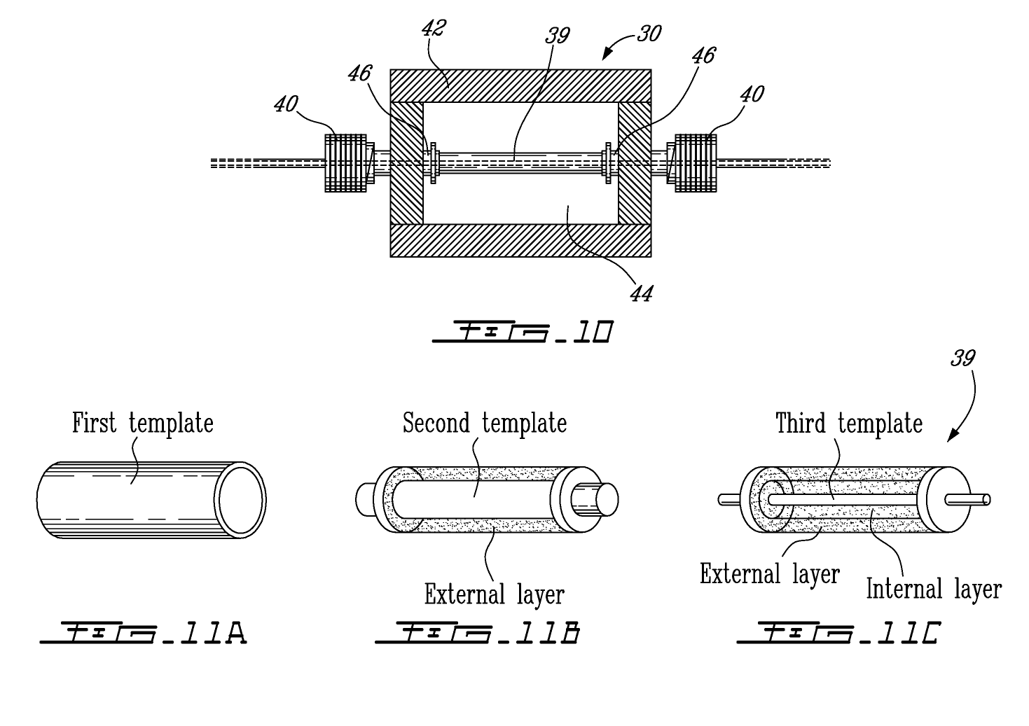

FIG. 12A shows a B-mode image (at 10 mmHg) of the phantom 39. To ensure the reproducibility of the method, VM elastograms (Equation 4) were computed comparing im₁ⱼ and im₂ⱼ (j=1, 2, 3, 4). FIG. 12B presents an average of 4 such elastograms that shows the visibility of both layers. FIG. 12C shows a graph of an average of 5 axial lines chosen in the middle of the VM elastogram (around x=0). Both layers can quantitatively be differentiated, specifically at the upper side of the vessel (left side of panel c) where maximum strains around 0.8% and 0.4% are observed for the inner and the outer layers, respectively.

As shown by Equation 4, the composite VM elastogram is obtained by computing the four components of the deformation matrix. Whereas the method 100 adapted to NIVE or MicroNive provides very accurate axial deformation estimates, lateral motion assessment remains a non-trivial problem, due to the relatively limited lateral resolution of RF ultrasound images. This difficulty was partially circumvented. Indeed, under the assumptions of weak compressibility of biological soft tissues and isotropy inside each measurement-window, the lateral strain (εₓₓ=Δₓₓ) was deduced from the axial strain (ε_{yy}=Δ_{yy}), that is Δ_{yy}=Δₓₓ/2.

From FIG. 12B, the strain in the inner layer close to the vessel lumen was on average 1.11±0.05%. Since the intraluminal pressure gradient was 5 mmHg, the elastic modulus E was estimated at 60±3 kPa for this material (made with two freeze-thaw cycles). It is to be noted that the elastic modulus E, for the inner layer, was estimated from Equation 5. Indeed, as a first approximation, a for this layer is given by the static pressure gradient inside the vessel measured for the conditions corresponding to the pre-motion and post-motion RF images. E has been estimated at around 49±6 kPa for a 1 freeze-thaw cycle PVA-C. In both cases, the pressure pre-load was 10 mmHg. The elastic modulus E was higher for the 2 freeze-thaw cycles material as it could be expected, since PVA-C made of 1 freeze-thaw cycle is softer than PVA-C made of 2 freeze-thaw cycles.

Even though the experimental set-up from FIG. 9 has been described including a high-resolution transducer, with a 32 MHz central frequency, to acquire the RF data for the in vitro experimentations of MicroNive, lower frequencies can also be used, for example 10 MHz or lower for in vivo NIVE applications to counteract ultrasound signal attenuation. Therefore a transducer is carefully selected for the system 10 since it is considered that, for ultrasound signals, lower is the frequency, deeper the beam propagates in the tissue, but, higher is the frequency, better is the resolution.

NIVE applications of the method 100 and of the system 10 include characterizing abdominal or peripheral aneurysms and superficial arteries such as the femoral and the carotid.

### In Vivo Experimentation of NIVE on the Carotid Artery of a Normal Human Subject

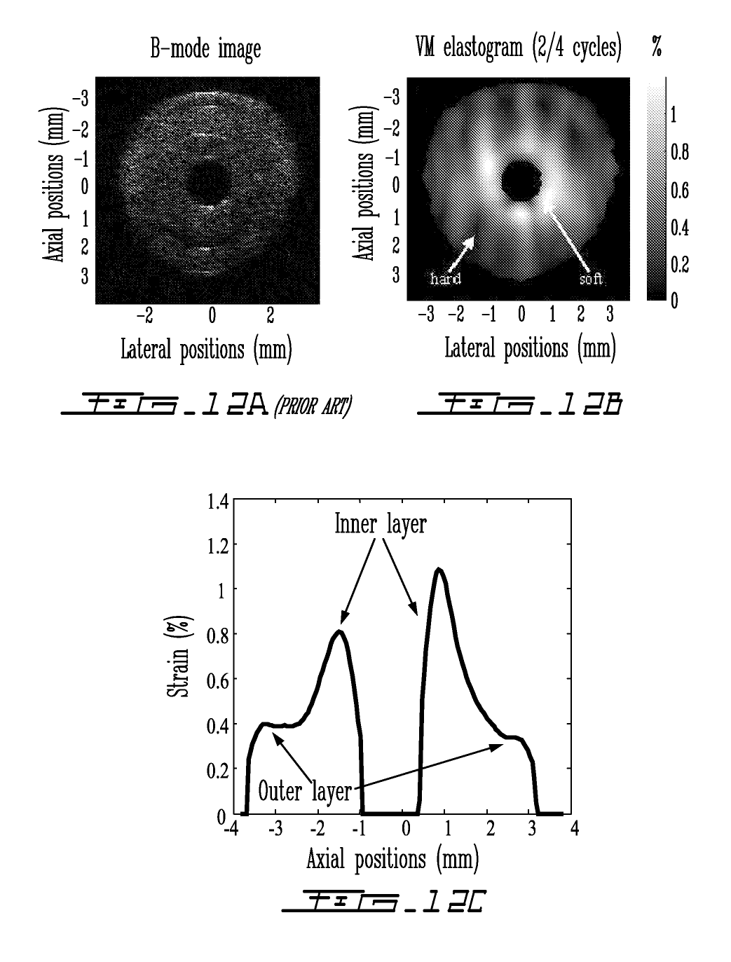

RF data were acquired from the carotid artery of a young healthy human subject. The Ultrasonix™ ES500RP ultrasound system, along with a 7.5 MHz transducer, was used to image longitudinal sections of the artery. FIG. 13A shows a B-mode image of the artery. FIG. 13B presents a manual segmentation of the same artery. FIGS. 13C-13D present axial strain elastograms computed with the method 100; the gray-scaled “colorbar” expresses the deformation in percentage. Since these elastograms were computed from data acquired during diastole, the axial strain values are expected to be positive. The regions of interest highlighted in FIGS. 13C and 13D correspond to sections of the carotids where motion occurred close to parallel to the ultrasound beam. Interestingly, in both elastograms, the upper vessel walls are observed to deform less than the lower walls; that is because the force exerted by the transducer over the skin can be seen as a boundary condition limiting the motion of the upper vascular tissues. It is to be noted that the Von Mises coefficient has not been used to display the strain patterns obtained from the method 100, because longitudinal sections of the carotid vessels were acquired instead of transverse planes.

In Vivo Experimentation of MicroNIVE on the Carotid Artery of normotensive and hypertensive rats

The method 100 can be used to characterize mechanical properties of small vessels (MicroNIVE) in humans or animals. More specifically, the method 100 can be used in the context of the phenotyping in hypertension (HT) with genetically-engineered rat models.

High-frequency ultrasound RF data were acquired on 6 male rats: 3 normotensive Norway Brun rats (labeled as NT1, NT2 and NT3) and 3 spontaneously hypertensive SHR rats (HT1, HT2 and HT3), respectively. All animals were 15-weeks old and they were anesthetized by inhalation of 1.5% isofluorane during RF data acquisition. The body temperature of each animal was monitored with a rectal probe and maintained at 37±1° C. by using a heating surface. The hairs over the neck were shaved and further removed with a depilatory cream. Sequences of RF data were acquired over longitudinal sections of the carotid artery using the Vevo 660™ from Visualsonics, an improved version of the VS-40 ultrasound system (that was used to acquire the in vitro phantom data reported above in FIG. 12). Indeed, this ultrasound system is equipped with an encapsulated oscillating element transducer and provides a frame rate of 30 images/s. For the purpose of the in vivo investigation, a 40 MHz central frequency transducer was used. The mean systolic pressures for NT and HT rats (measured with a tail-cuff monitoring system) were 87±12 mmHg and 158±16 mmHg, whereas the heart beats were 323±15 beats/min. and 365±6 beats/min., respectively.

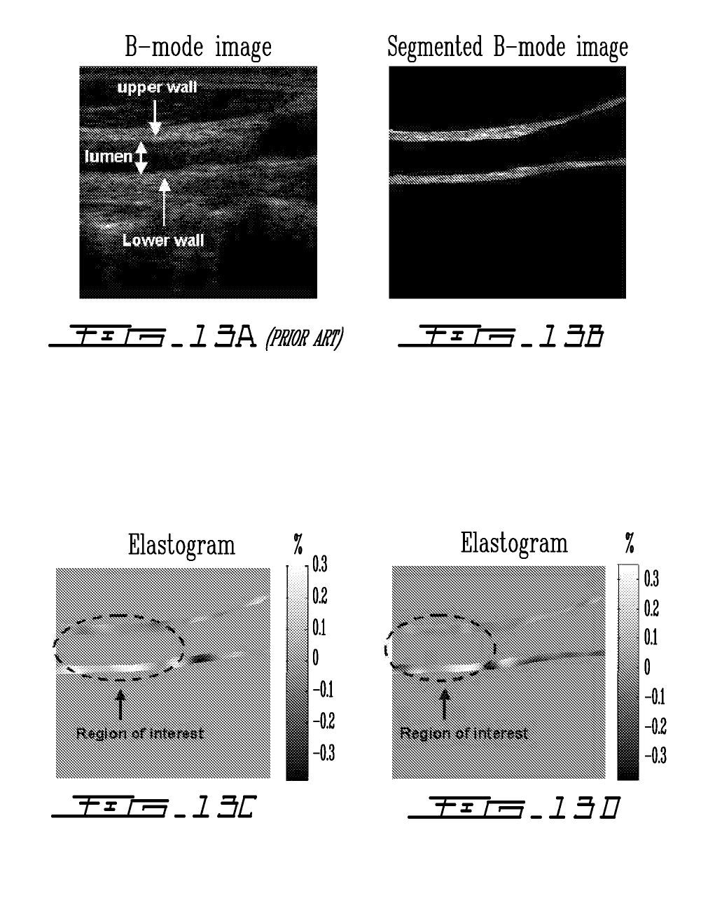

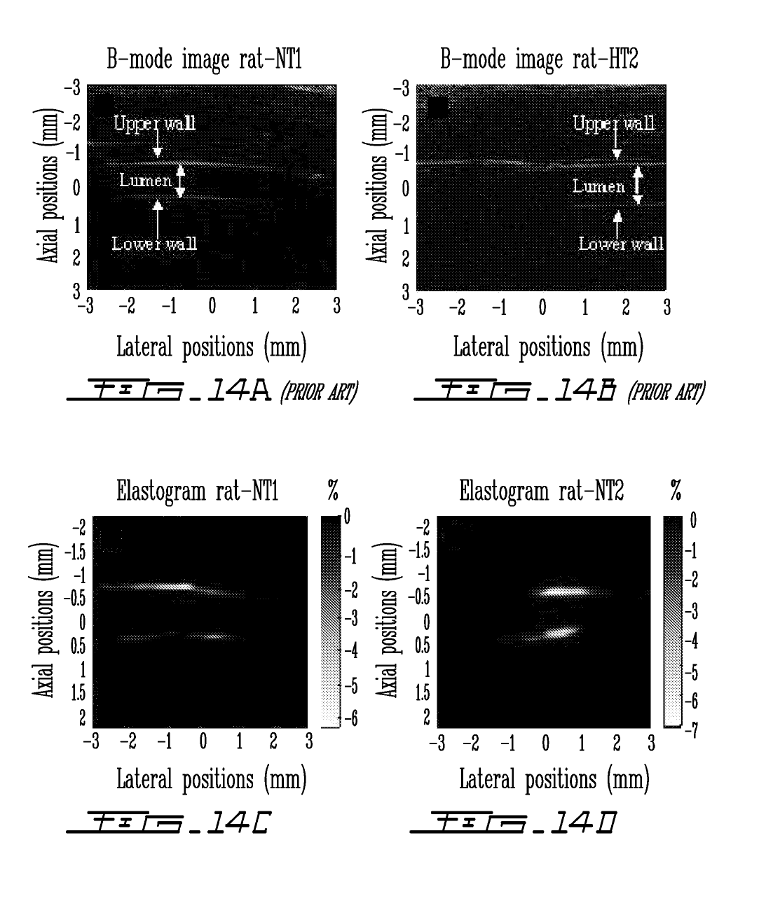

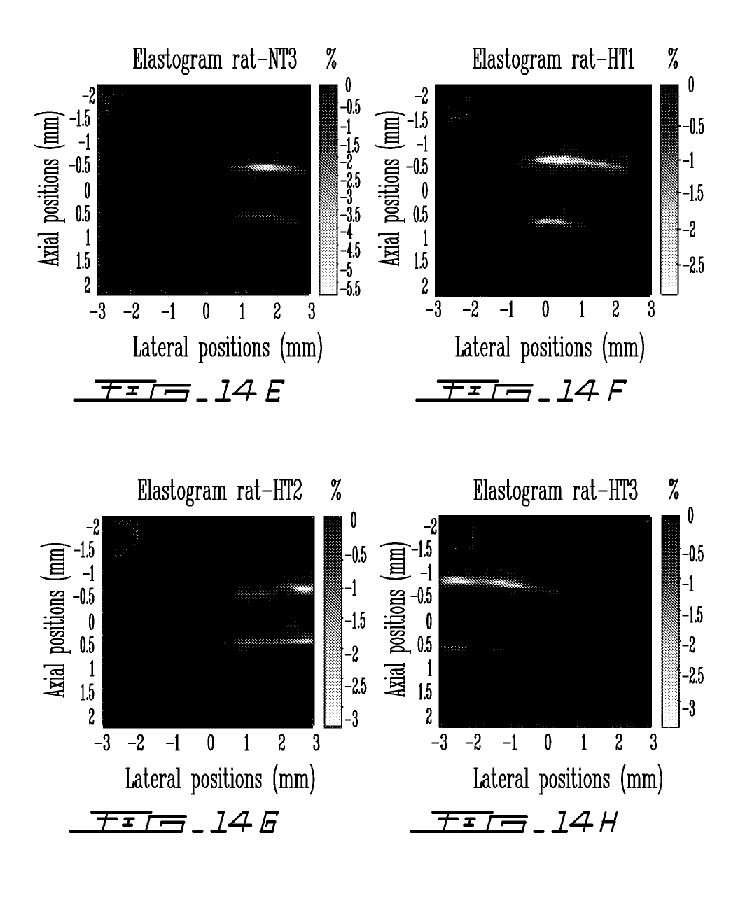

Elastograms were computed using the method 100 adapted for MicroNIVE as it will now be described. All successive acquired RF images that were digitized over several cardiac cycles were used. No averaging was used to display the axial elastograms of FIGS. 14C-14H. Manual segmentation has been done to display only the strain patterns within the vascular wall. It is to note that the Von Mises coefficient has not been used to display the strain patterns obtained from the method 100, because longitudinal sections of the carotid vessels were acquired instead of transverse planes.

FIGS. 14A-14B show two B-mode images obtained for a normotensive rat (NT1) and a hypertensive one (HT2), respectively. In both cases, the internal diameter of the carotid was around 1.1 mm, whereas the wall thickness was close to 160 μm for NT1 and 120 μm for HT2, respectively.

FIGS. 14C-14H show axial strain elastograms computed using the method 100; the gray-scale “colorbar” providing the strain in percent. The negative strains are indicative of vessel dilation (diastolic phase). Since it has been difficult, for most rats, to have longitudinal sections of 6 mm, only portions of the carotids are displayed on the elastograms. The carotids of the three normotensive rats (NT1, NT2 and NT3) appear on average twice softer (strain values up to 7%) than those of the hypertensive ones (HT1, HT2 and HT3), where a maximum of 3.3% strain was estimated. To provide a more rigorous interpretation of these data, one should of course consider the pressure gradient between the pre-compression (pre-tissue-motion) and post-compression (post-tissue-motion) RE images used to compute the elastograms. This pressure gradient was higher for HT than NT rats. However the exact values are not known since the RF images were acquired without any ECG or pressure synchronization.

A method and system for MicroNIVE according to the present invention can be used in ex-vivo experiments or in vivo testing on animals or humans. For example, using recombinant inbred strain (RIS) rats, the method can be used to examine the modulation of drug-induced cardiovascular remodeling as a function of HT and aging. Examples of protocols for ex-vivo and in vivo experiments are described in the following.

Animals are treated with placebo, losartan, which is an antihypertensive drug and an antagonist of angiotensin II (ANG II) type 1 (AT1) receptors (30 mg/day), and nifedipine, an antihypertensive drug, which is a calcium channel blocker (30 mg/day) for two weeks starting at 12 weeks of age. Regarding the ex-vivo experiments, the animals are killed and segments of arteries (carotid, for example) are excised. Segments (≈2-cm in length) will be mounted on similar apparatus than for the vessel-mimicking phantom experimentation described above in FIGS. 9 and 10. Examples of preparation and of protocol of study for such arteries are well documented [Li et al., 1998 and 2003; Intengan et al., (1998a and 1998b)]. The vessel is adjusted to its length before excision such as the vessel walls become parallel. The vessel is equilibrated under a constant intraluminal pressure of 45 mmHg with physiological salt solution [Intengan et al., (1998a and 1998b)]. A servocontrolled pump stepwise (5 mmHg each step) increases the intraluminal pressure, and time-sequence RF data are acquired at different frequencies (25 or 40 MHz, depending on the artery) with an ultrasound biomicroscanning system, such as the Vevo 660™ from Visualsonics. The elastograms are computed using a method for vascular elastography according to the present invention, such as the method 100.

Other in vivo experiments can also be performed using RIS rats. These animals are treated with placebo, losartan (30 mg/day), and nifedipine (30 mg/day) for two weeks starting at 12 weeks of age for the purpose of examining the modulation of drug-induced cardiovascular remodeling as a function of HT and aging. To acquire RF data, the rats are anesthetized by inhalation with 1.5% isofluorane. Physiological parameters (temperature, pressure and ECG) are monitored. The temperature is maintained close to 37° C. using a hot plaque. The region of interest is shaved using a conventional'electric shaver; the remaining hair is removed with Nair™ or another lotion hair remover. Even though there is possible impact of anesthesia on the cardiac function of rats (reduction of the heart beat, cardiac output, etc. . . . ), and possibly on the arteries, that effects is the same for all animals and therefore does not interfere with the interpretation of the results since all the rat strains are anesthetized.

In the case where carotid arteries are investigated, only the axial strain are required to study longitudinal images, since the vessel wall motion can be seen as running parallel with the ultrasound beam. The RF data are processed using the method 100 to provide step-wise elastograms (strain images). From the strain estimates, another mechanical parameter (namely stress/strain ratio) is calculated.

The MicroNIVE method according to the present invention allows providing significant new insights regarding the pathophysiology of HT and aims at leading to new discoveries in the field of pharmacology for example, even though it is not limited to this particular application.

### Simulated and Experimental In Vitro Results on Endovascular Elastography (EVE)

A method for endovascular elastography (EVE) according to a third illustrative embodiment of the second aspect of the present invention will now be described. Since the method according to this third embodiment is similar to the method illustrated in FIG. 2, and for concision purposes, only the differences between these two methods will be described herein.

The first step of the method is to acquire intravascular RF images using a catheter. Following the example of IVUS, and as schematically illustrated in FIG. 15, a transducer is placed at the tip of the catheter and cross-sectional imaging of a vessel is generated by sequentially sweeping the ultrasound beam over a 360° angle. It is to be noted that, in the ideal situation illustrated in FIG. 15, the ultrasound beam runs parallel with the vascular tissue motion, i.e. in the (r,φ) coordinate system.

Mechanical parameters (radial strain, in this case) are then estimated from analyzing the kinematics of the vascular tissue during the cardiac cycle or in response to an angioplasty-balloon push or to any other force exerted axially onto the inner vascular wall.

The Lagrangian Speckle Model Estimator (LSME) is then formulated for investigations in EVE, i.e. using a polar coordinate system. Indeed, while the full 2D-deformation matrix Δ can be assessed, only the radial strain component Δᵣᵣ (=εᵣᵣ) is displayed. This is motivated by the fact that tissue motion, in EVE, is expected to run close to parallel with the ultrasound beam. Again, the LSME allows computing the full deformation matrix Δ of Equation 3. Since Δ is directly assessed, no derivative of the displacement fields is required, as it was also the case for the first and second embodiments of the present invention. Although the method is general and applies to either 1D, 2D or 3D RF data, the description given hereafter refers to a 2D model for simplicity.

In 2D, using polar coordinates, the LSME can be formulated as:

$$\begin{matrix}\begin{matrix}MIN \\ {LT}_{p}\end{matrix} {‖ \begin{matrix}I ( r , ϕ , 0 ) - \\ {[ I ( r , ϕ , t ) ]}_{{LT}_{p}}\end{matrix} ‖}^{2} = \begin{matrix}MIN \\ {LT}_{p}\end{matrix} {‖ I ( r , ϕ , 0 ) - {I}_{Lag} ( r , ϕ , t ) ‖}^{2} \\ = \begin{matrix}MIN \\ {LT}_{p}\end{matrix} {‖ ℜ ( r , ϕ , t ) ‖}^{2}\end{matrix} \qquad ( 14 )$$

The minimum is obtained by using the appropriate [LTₚ]. [LTₚ] is a linear transformation matrix which maps the Cartesian trajectories in a polar coordinate system. However, for a small ROI (Δr, Δφ) that is far from the vessel lumen center, motion equivalently can be investigated using either a polar or a Cartesian coordinate system. In other words, the following approximation can be done to compute the elastogram:

ξ=LT−I≅LTₚ−I (15)

where I is the 2D-identity matrix. In other words, the solution to Equation 13 can be obtained from solving Equation 6.

### Biomechanical Simulations of Vessel Wall Kinematics

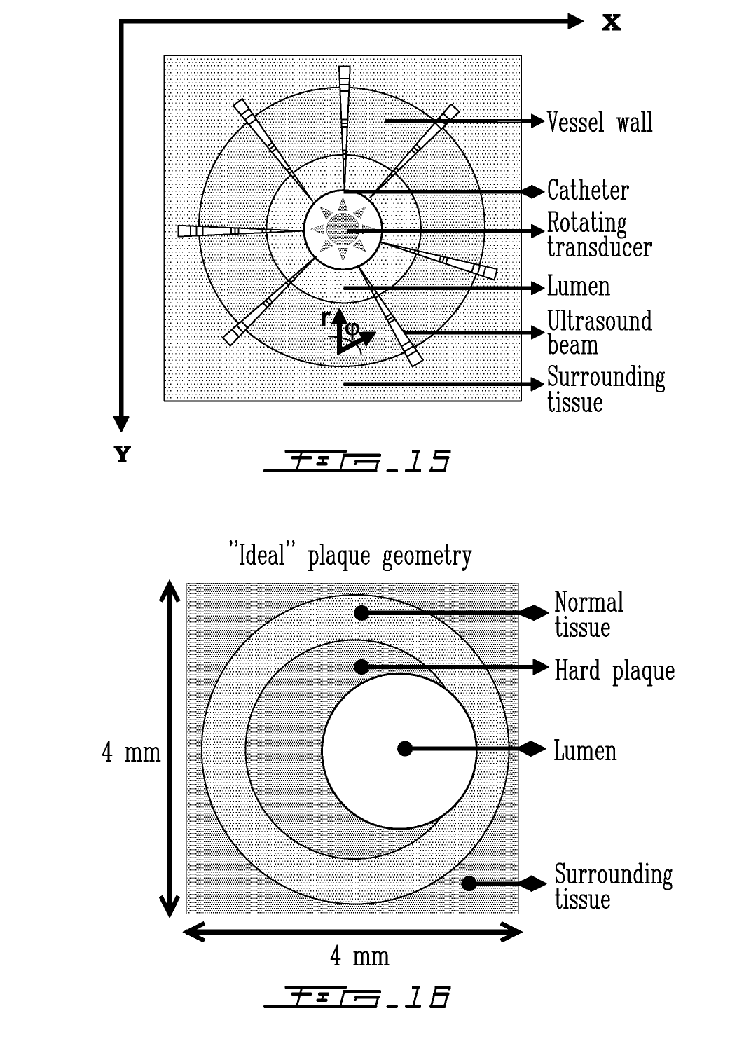

A computational structural analysis has been performed on one simulated idealized coronary plaque (see FIG. 16) and on a model identified on FIG. 17B created from measurements made of a typical composite plaque identified from an in vivo IVUS image of a patient with coronary artery disease (see FIG. 17A). The former allowed validating the potential of the EVE method according to the present invention to differentiate between hard and soft vascular tissues and the latter allowed characterizing the heterogeneous nature of atherosclerotic plaques, which is linked to the risk of rupture and thrombosis.

For the two models, the materials were considered as quasi-incompressible (Poisson ratios v=0.49) and isotropic with linear elastic properties. The Young's modulus for the healthy vascular tissue (or adventitia & media) was 80 kPa [Williamson et al., (2003)], while the dense fibrosis (much stiffer) was set at 240 kPa, and the cellular fibrosis (softer than the dense fibrosis) was chosen at 24 kPa [Ohayon et al., (2001); Treyve et al., (2003)]. Whereas the surrounding tissue was not investigated, the bulk boundary conditions, as it may eventually be provided by surrounding organs, were simulated by imbedding the vessel in a stiffer environment of 1000 kPa Young's modulus.

Finite element (FE) computations were performed by considering static simulations of coronary plaques under loading blood pressure. The simulations were performed on the geometrical models previously described (see FIGS. 16 and 17B). Nodal displacements were set to zero on the external boundaries of the surrounding tissue. The various regions of the plaque components were then automatically meshed with triangular (6 nodes) and quadrangular (8 nodes) elements. The FE models were solved under the assumption of plane and of finite strains. The assumption of plane strain has been made because axial stenosis dimensions were of at least the same order of magnitude as the radial dimensions of the vessel. Moreover, the assumption of finite deformation was required as the strain maps showed values up to 30% for physiological pressures [Loree et al., (1992); Cheng et al., (1993); Lee et al., (1993); Ohayon et al., (2001); Williamson et al., (2003)]. However, the kinetics reported were achieved with small pressure gradients (around 15 mmHg) such that the radial strain remained below 10%. The Newton-Raphson iterative method with a residual nodal tolerance of 4×10⁻⁴ N was used to solve the FE models. The calculations were performed with a number of elements close to 7200. This computational structural FE analysis was used to perform the kinematics of the vascular tissue.

A dynamic image-formation model was implemented using the Matlab™ software. It is assumed that the image formation can be modeled as a linear space-invariant operation on a scattering function, and that the motion occurs in plane strain conditions (such as no transverse deformation is involved). It considers a scattering function Z(x(t₀),y(t₀)) (at t₀=0) that simulates the acoustical characteristics of a transverse vascular section in Cartesian coordinates. Knowing the axial and lateral displacement fields, they are applied upon Z(x(t₀),y(t₀)) to perform tissue motion, thus providing Z(x(t),y(t)). The last step consists of convolving Z(x(t),y(t)) with the PSF (point-spread-function) to provide a dynamic sequence of RF images I(x(t),y(t)) or equivalently I(x,y,t). The PSF is the equivalent image of a single cellular ultrasound scatterer. In other words, the PSF expresses the intrinsic characteristics of the ultrasound imaging system. It can be determined experimentally by using a phantom (a box containing a tissue-mimicking gel) containing a point target. The dynamic image-formation is of interest to simulate the RF data.

The idealized vessel illustrated in FIG. 16 measured about 3.8 mm in outer diameter, whereas the RF images extended to 4 mm×4 mm. The real case vessel illustrated in FIG. 17B measured about 7 mm in outer diameter, whereas the RF images extended to 8 mm×8 mm. For the purpose of simulations, the intraluminal pressure gradients. were set at 15.79 mmHg and 11.73 mmHg for the idealized and the realistic vessels, respectively. Considering the above, the dilation at the inner wall was around 7% in both cases. The PSF characterized a 20 MHz central frequency IVUS transducer. The LSME was implemented to assess tissue motion. Measurement-windows of 0.38 mm×0.40 mm and 0.77 mm×0.80 mm, with 90% axial and lateral overlaps, were used for the idealized and the realistic cases, respectively.

### Investigation of the “Ideal” Plaque Pathology

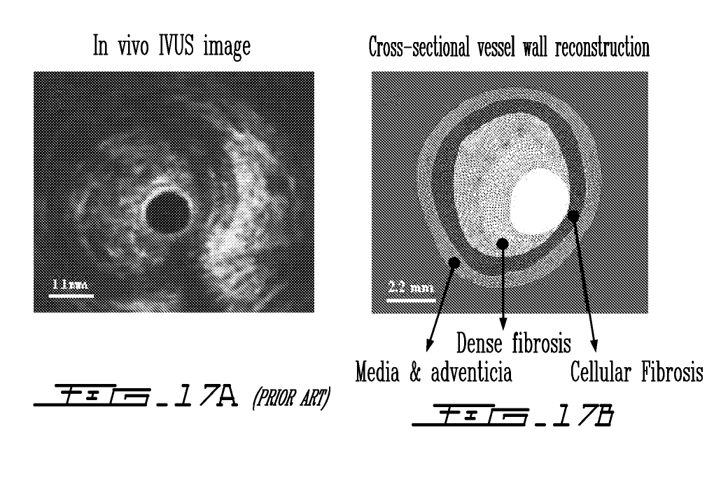

FIG. 18A presents the theoretical radial strain elastogram, computed for the “ideal” pathology case, using Ansys FE and Matlab softwares. The plaque can slightly be differentiated from the normal vascular tissue, whereas a region of higher strain values is observed at the right portion of the inner vessel wall. This “mechanical artifact” is a direct consequence of the well known strain decay phenomenon [Shapo et al., (1996a)]. For a more quantitative illustration, are presented in FIG. 18B plots from the theoretical elastogram for two orthogonal orientations along x and y. Indeed, the vertical plot (—) shows low contrast between the plaque and the normal vascular tissue, whereas the horizontal plot (---) clearly points out the presence of strain decay.

FIG. 18C presents the radial strain elastogram as computed using the EVE method from the present invention, using simulated RF images. As for the theoretical elastogram in FIG. 18A, the plaque is slightly distinguishable from the normal vascular tissue. The graphs of FIG. 17D confirm such an observation.

As illustrated in FIGS. 18A and 18C, the present invention allows both characterizing the strain in the vessel quantitatively in addition to qualitatively. Indeed, the gray-scale “colorbars” at the right of each Figure express the strain in percent.

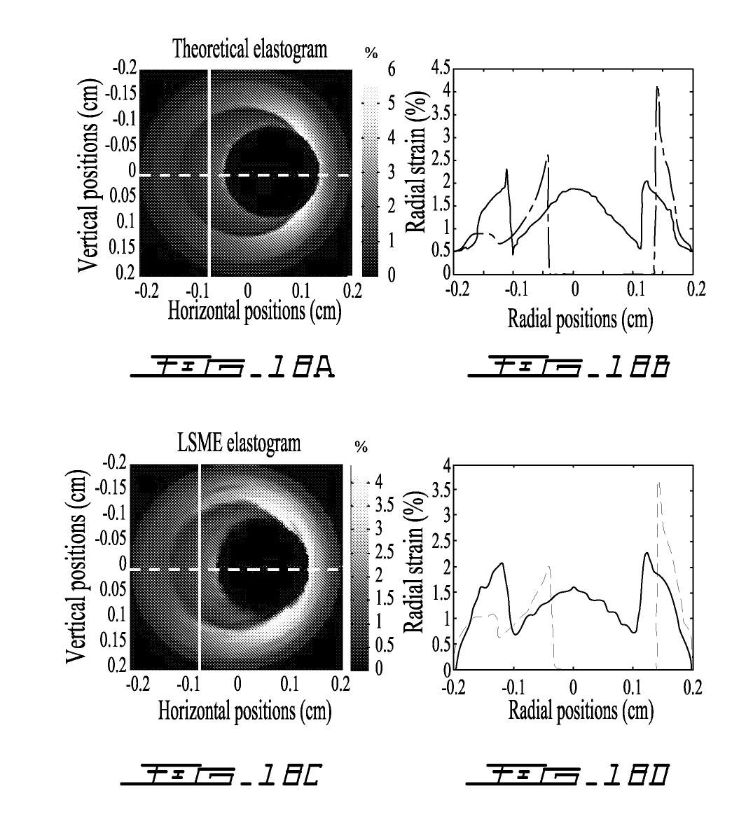

For the purpose of compensating for strain decay, the radial strain elastogram resulting from the method according to the present invention was post-processed. Indeed, εᵣᵣ was modulated with a function proportional to the square of the vessel radius. The strain-decay-compensated elastogram issued from the EVE method according to the present invention is represented in FIG. 19A and shows substantial contrast improvement. For instance, the axial plot of FIG. 19B shows an effective contrast ratio close to 3 between the plaque and the normal vascular tissue, as it can be expected. Equivalently, FIG. 19C also shows some valuable contrast ratio improvement compared to FIG. 18D.

### Investigation of “Realistic” Vessel Wall Pathology

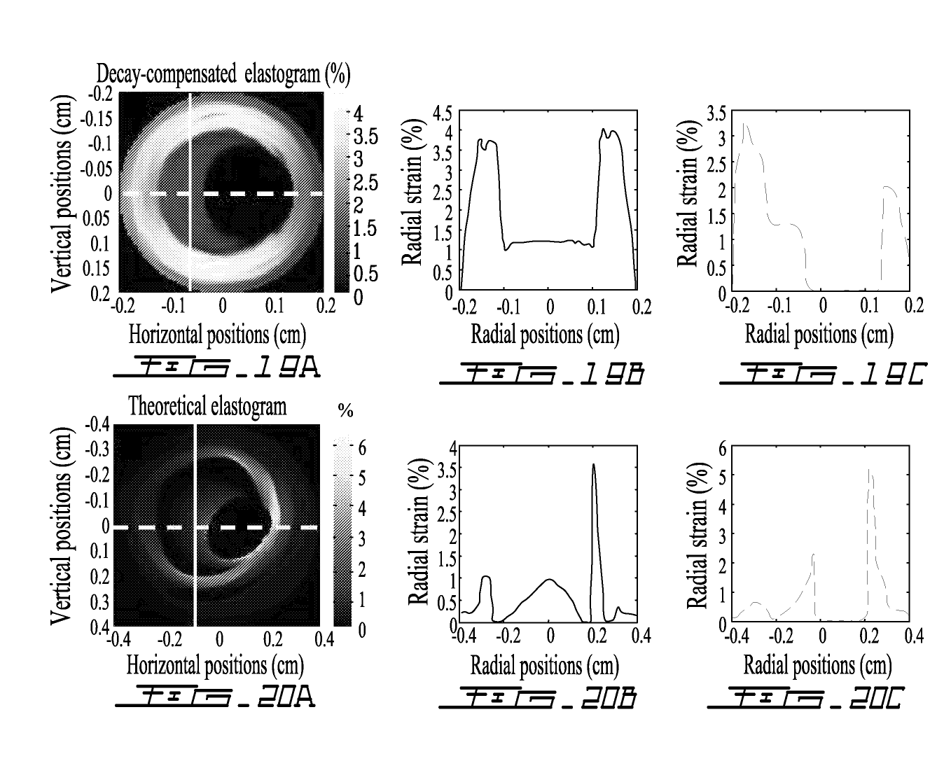

FIG. 20A illustrates the theoretical radial strain elastogram, computed for the “realistic” pathology case. Interestingly, complex strain patterns are observed; nevertheless, different regions can be identified. For instance, since the ratio of Young's moduli between the dense and the cellular fibroses was set to 10, both of those materials can be distinguished. Less contrast is seen between the cellular fibrosis and the healthy vascular tissue because their Young's modulus contrast was set to 3. As illustrated with vertical and horizontal 1D plots from the elastogram (FIGS. 20B and 20C, respectively), strong strain decay is observed specifically at the inner portion of the vessel wall.

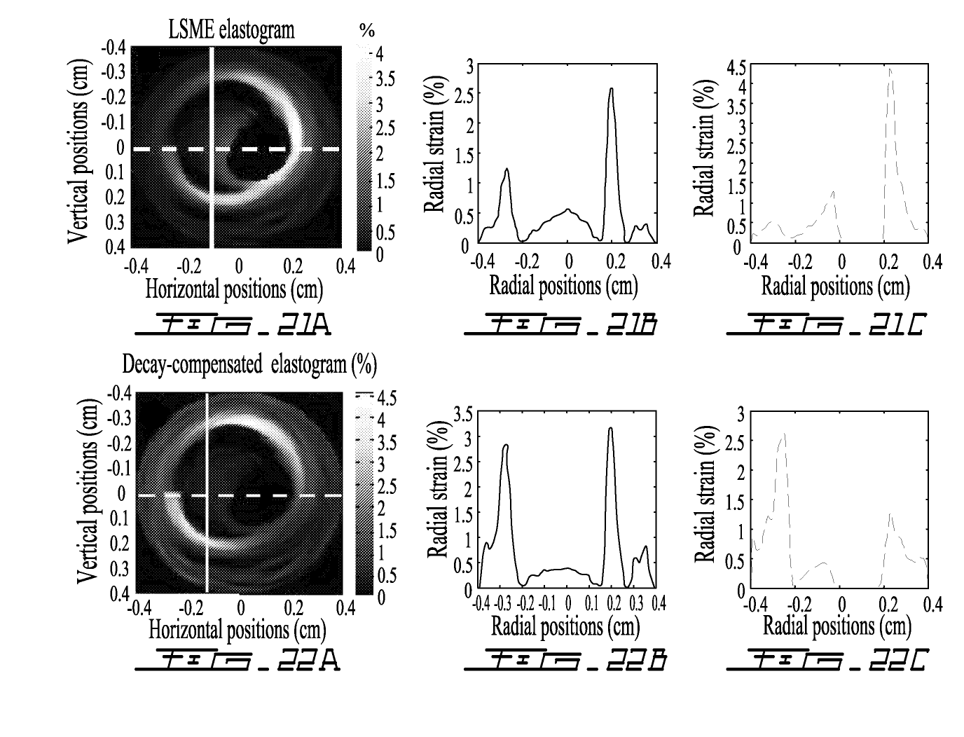

FIG. 21A illustrates the radial strain elastogram as computed using the method for endovascular elastography according to the third illustrative embodiment of the second aspect of the present invention, using simulated RF images. Comparing to the theoretical elastogram in FIG. 20A, very complex strain patterns are also observed. Moreover, the dense and the cellular fibrosis tissues can be identified. However, while less prominent than in the “ideal” case study, strain decay remains a significant factor to compensate for to improve image interpretation. This is illustrated in FIGS. 21B and 21C, where vertical and horizontal 1D graphs from the elastogram are presented.

FIG. 22A illustrates the strain-decay-compensated LSME elastogram, showing substantial contrast improvement. Both the vertical graph (FIG. 22B) and the horizontal one (FIG. 22C) show more effective contrast ratio between dense and cellular fibroses, and between cellular fibrosis and the normal vascular tissue. Moreover, it is interesting to notice the presence of moderate strain values (around 0.6 to 0.8%) at the extremities of the plots; this characterizes regions of healthy vascular tissue, namely the media and adventitia.

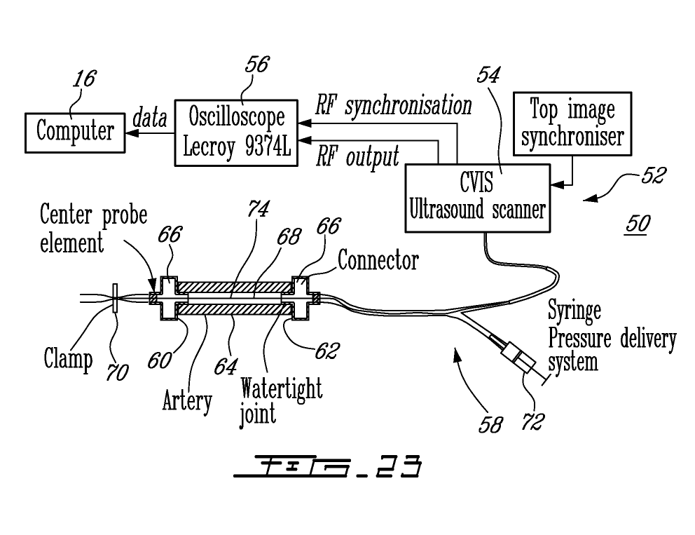

The method for endovascular elastography according to the present invention has also been validated in vitro using a fresh excised human carotid artery. The experimental set-up 50 used in the validation is illustrated in FIG. 23. The set-up 50 includes a system 52 for endovascular elastography according to a second embodiment of the first aspect of the present invention.

The system 52 comprises an ultrasound scanner 54 in the form of a CVIS (ClearView, CardioVascular Imaging System Inc.) ultrasound scanner, working with a 30 MHz mechanical rotating single-element transducer (not shown), a digital oscilloscope 56, more specifically the model 9374L from LECROY, and a pressuring system 58.

The extremities 60-62 of an artery 64 are fixed to two rigid sheaths by watertight connectors 66, separated according to the original longitudinal dimension of the vessel 64 before excision. The intravascular, catheter 68, part of the system 52 was introduced through the proximal sheath into the lumen of the artery 64, and then through the distal sheath. The distal sheath was closed with a clamp 70 to insure watertightness of the system 58. Injecting fluid inside the system 58 resulted in an increase of the pressure inside the arterial lumen since the sheath is rigid and the system is watertight. A syringe 72 is then connected to the proximal sheath and the inner pressure is increased or decreased by manually varying the fluid volume (precision: ΔV=0.01 ml) inside the lumen. Whereas the quantitative pressure values were not monitored by an independent means, the fluid volume inside the lumen was maintained constant during each acquisition of RF ultrasound data.

The ultrasound probe 74 was fixed approximately at the center of the arterial lumen by two guiding elements. This protocol was used to limit probe motion and accordingly to reduce geometrical artifacts [Delachartre et al. (1999)].

A sequence of radio-frequency (RF) images was collected while incrementally adjusting the intraluminal static pressure steps. At each static pressure step (volume step), a scan of 256 angles was performed. A set of 11 RF images was so acquired for consecutive increasing physiologic fluid pressure levels. Sampling of the data was phase-synchronized, with the top image synchronizer and the RF signal synchronization (external outputs of the CVIS ultrasound scanner). The top image synchronizer allows the user to select an angular position from which the acquisition started; it thus permitted the acquisition of sets of images angularly aligned. The RF signal synchronization was done at the pulse repetition frequency of the bursts transmitted to the single-element transducer. RF data were digitized at a 500 MHz sampling frequency in 8 bits format, stored on a PCMCIA hard disc in the LeCroy oscilloscope and processed off line.

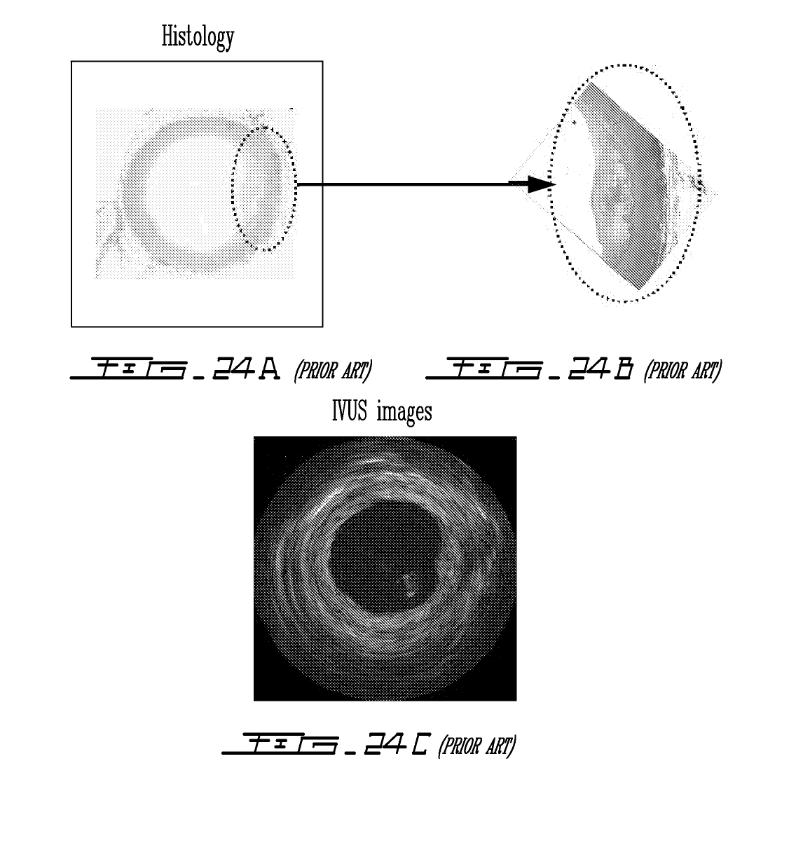

As shown by histology (FIGS. 24A and 24B), the artery was characterized by a thin atherosclerotic plaque (located at about 3 o'clock), that was only restricted to a confined angular sector. The coloration with saffron haematoxylin-eosin revealed that the plaque contained cholesterol crystals and inflammatory cells. It is to be noted that the IVUS image on FIG. 24C does not clearly allow differentiating the plaque from the healthy vascular tissue and therefore appears insufficient to characterize vascular tissue.

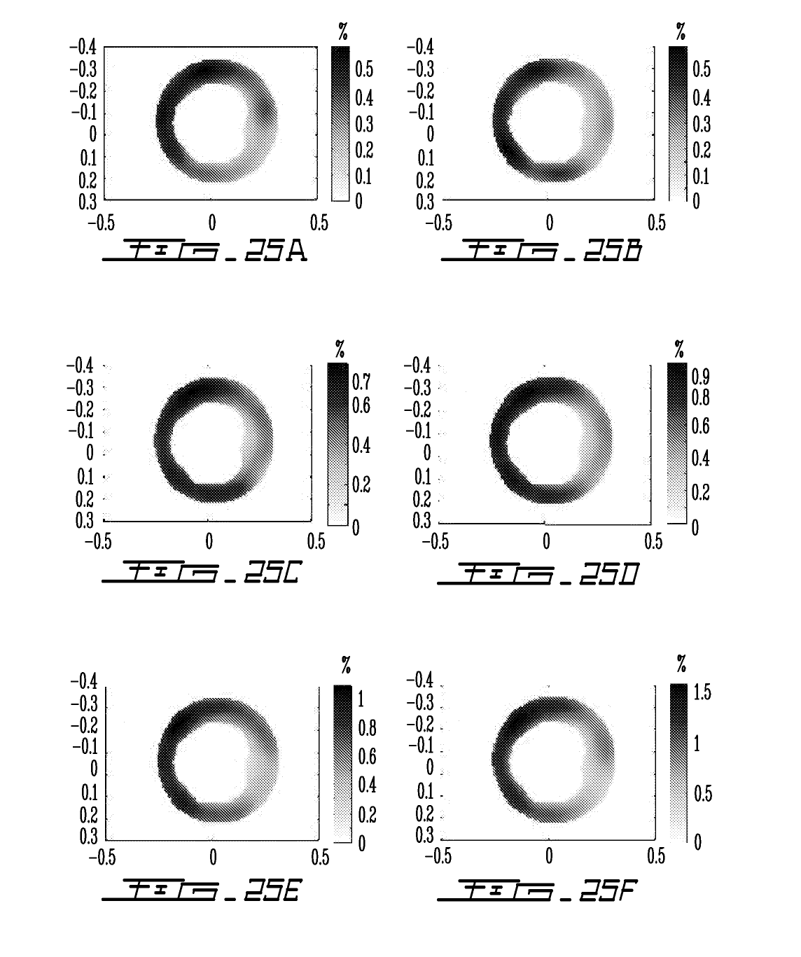

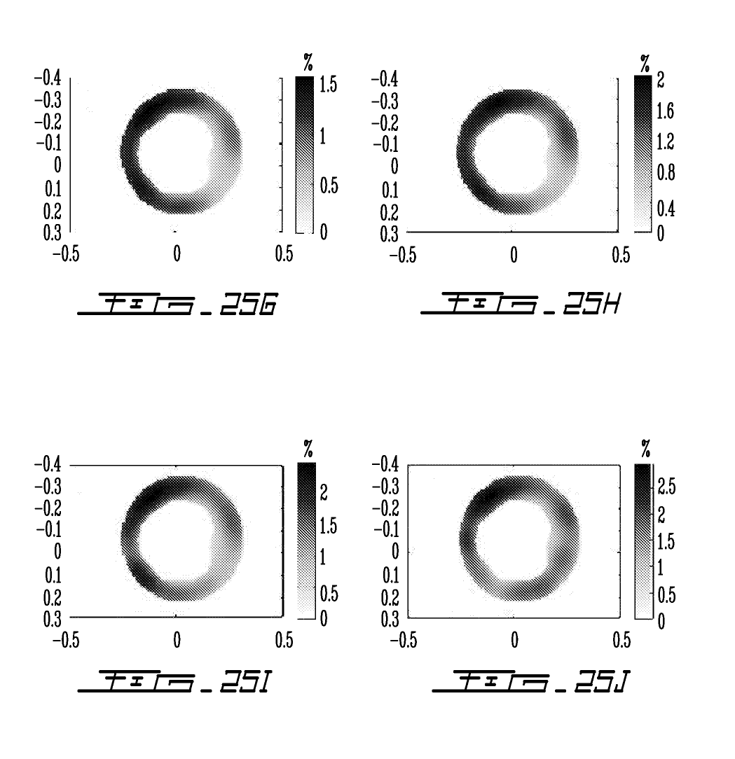

FIGS. 25A-25J show 10 radial elastograms that were computed, using the set of 11 RF images acquired for consecutive increasing physiologic fluid pressure levels using the method for endovascular elastography according to the present invention.

The elastogram obtained for the lowest intraluminal pressure (i.e. from the 1^{st} and 2^{nd} RF images, in this case) is displayed in FIG. 25A, whereas FIG. 25J shows the elastogram for the highest pressure difference (i.e. the elastogram computed with the 1^{st} and 11^{th} RF images). Indeed, maximum strain values close to 0.6% are observed in FIG. 25A, whereas the maximum is close to 3% in FIG. 25J. To summarize, elastograms in FIGS. 25A and 25J are the least representative, and those from FIG. 25C to FIG. 25E present very good plaque detectability, accuracy in plaque dimensions, and significant contrast between plaque and surrounding tissue. This demonstrates that a range of intraluminal pressures for which tissue motion estimation appears optimal exists.

It is to be noted that, in the elastograms from FIGS. 25A-25J, lateral and axial values are dimensions in centimeters, while the gray-scaled “colorbars” give the strain in percent.

The above results showed the potential of the method for endovascular elastography according to the present invention to characterize and to distinguish an atherosclerotic plaque from the normal vascular tissue. Namely, the geometry as well as some mechanical characteristics of the detected plaque are in good agreement with histology. The results also suggested that there might exist a range of intraluminal pressures for which plaque detectability is optimal. The plaque at 3 o'clock displayed low strain values indicative of a stiff tissue.

The EVE method according to the present invention further allows providing quantitative parameters to support clinicians in diagnosis and prognosis of atherosclerotic evolution.

Furthermore, regarding the above experimental results of the application of the EVE method to characterize human artery, while an optimal range of intraluminal pressures seems to be indicated to improve plaque detectability, the results also showed two specific features. Firstly, comparing elastography with histology, the geometry of the plaque appears to be preserved in the LSME elastograms (see FIGS. 25C and 25D). For instance, the maximum plaque thickness measured in the elastogram of FIG. 25C is close to 360 μm; this estimation is strongly supported by histology measurement conducted by Brusseau et al. (2001), who found a maximum plaque thickness of approximating 350 μm for this very same carotid artery segment. Secondly, regarding biomechanical properties, a strain ratio close to 3 could be observed between the atherosclerotic plaque and the healthy surrounding vascular tissue for all elastograms presented in FIGS. 25. Such information may provide interesting insights about plaque components; it thus may help predicting plaque rupture and also help in therapy planning. The possibility of assessing quantitative strain values with the LSME represents an advantage of the present EVE method over the prior art.

A major advantage of the present EVE method over correlation-based techniques [de Korte et al., (1997; 1998; 2000a;) Brusseau et al., (2001); Talhami et al., (1994); Ryan and Foster, (1997); Shapo et al., (1996a)] stems from the fact that it allows computing the full strain tensor. For instance, complex tissue deformations such as rotation, scaling and shear can appropriately be assessed, whereas they are known to set a potential limitation for correlation-based methods.

Although the present invention has been described hereinabove by way of preferred embodiments thereof, it can be modified without departing from the spirit and nature of the subject invention, as defined in the appended claims.

### REFERENCES

Bang J., Dahl T., Bruinsma A., Kaspersen J. H., Hernes T. A. N., Myhre H. O. (2003), “A new method for analysis of motion of carotid plaques from RF ultrasound images”, Ultrasound in Med. & Biol., Vol. 29, No. 7, pp. 967-976.
Brusseau E., Fromageau J., Finet G., Delachartre P., and Vray D. (2001), “Axial strain imaging of intravascular data: Results on polyvinyl alcohol cryogel phantoms and carotid artery”, Ultrasound in Medicine & Biology, Vol. 27, No. 12, pp. 1631-1642.
Cantini C., Kieffer P., Gorman B., Liminana P., Atkinson J., Lartaud-Idjouadiene I. (2001), Aminoguanidine and aortic wall mechanics, structure, and composition, Hypertension, Vol. 38, pp. 943-948.
Cheng, G. C., Loree, H., M., Kamm, R. D., Fishbein, M. C., Lee, R. T. (1993), “Distribution of circumferential stress in ruptured and stable atherosclerotic lesions: a structural analysis with histopathological correlation,” Circulation, Vol. 87, No. 4, pp. 1179-1187.
Chu K. C. and Rutt B. K. (1997), “Polyvinyl alcohol cryogel: An ideal phantom material for MR studies of arterial flow and elasticity”. Magn. Reson. in Med., Vol. 37, pp. 314-319.

Davies J. M. and Thomas A. C. (1985), “Plaque fissuring: The cause of acute myocardial infarction, sudden ischaemic death, and crescendo angina”, Br. Heart J., Vol. 53, pp. 363-373.
de Korte C. L, Céspedes E. I., Van der Steen A. F. W., and Lancée C. T. (1997), “Intravascular elasticity imaging using ultrasound—Feasibility studies in phantoms”, Ultrasound in Medicine & Biology, Vol. 23, No. 5, pp. 735-746.
de Korte C. L., Van der Steen A. F. W., Céspedes E. I., and Pasterkamp G. (1998), “Intravascular ultrasound elastography in human arteries: Initial experience in vitro,” Ultrasound in Medicine & Biology, Vol. 24, No. 3, pp. 401-408.
de Korte C. L., Pasterkamp G., Van der Steen A. F. W., Woutman H. A., and Born N. (2000a), “Characterization of plaque components with intravascular ultrasound elastography in human femoral and coronary arteries in vitro”, Circulation, Vol. 102, No. 6, pp. 617-623.
de Korte C. L., Van der Steen A. F. W., Céspedes E. I., Pasterkamp G., Carlier S. G., Mastik F., Schoneveld A. H., Serruys P. W., and Born N. (2000b), “Characterization of plaque components and vulnerability with intravascular ultrasound elastography,” Physics in Medicine & Biology, Vol. 45, No. 6, pp. 1465-1475.
Delachartre P., Cachard C., Finet G., Gerfault L., Vray D. (1999), “Modeling geometric artefacts in Intravascular Ultrasound Imaging”, Ultrasound in Medicine and Biology, Vol. 25, No. 4, pp. 567-575.
Falk E. (1989), “Morphologic features of unstable atherosclerotic plaques underlying acute coronary syndromes”, Am. J. Cardiol., 63(suppl E), pp. 114E-120E.
Falk E. (1992), “Why do plaques rupture?”, Circulation, Vol. 86(suppl. III), pp. 30-42.
Fisher A., Gustein D. E., Fayad Z. A., Fuster V. (2000), “Predicting plaque rupture: enhancing diagnosis and clinical decision-making in coronary artery disease”, Vasc. Med., Vol. 5, pp. 163-172.
Foster F. S., Zhang M. Y., Zhou Y. Q., Liu G., Mehi J., Chérin E., Harasiewicz K. A., Starkoski B. G., Zan L., Knapik D. A., and Adamson S. L. (2002), “A new ultrasound instrument for in vivo microimaging of mice,” Ultrasound in Med. & Biol., Vol. 28, No. 9, pp. 1165-1172.
Fung Y. C. Biomechanical Properties of Living Tissues. 2^{nd} Ed., New York: Springer Verlag, 1993, p. 568.
Garra B. S., Céspedes E. I., Ophir J., Spratt S. R., Zuurbier R. A., Magnant C. M., and Pennanen M. F. (1997), “Elastography of breast lesions: initial clinical results”, Radiology, Vol. 202, No. 1, pp. 79-86.
Goud C., Pitt B., Webb R. C., Richey J. M. (1998), “Synergistic actions of insulin and troglitazone on contractility in endothelium-denuded rat aortic rings”, Am. Physiol. Soc., Vol. 275 (Endocrinol. Metab. 38), pp. E882-E887.
Hamet P., Pausova Z., Tremblay J., Deng A. (2002), “Hypertension as a genetic disease”, In: Hypertension Manual, Chapter 2.2, Mancia G., Chalmers J., Ferrari A., Julius S., Saruta T., and Weber M., Eds, Harcourt Brace and Co. Ltd, Robert Stevenson House, Scotland, UK.
Hinze, J. O. Turbulence. 2^{nd} Ed., New York: McGraw-Hill, 1975, pp. 1-781.
Horn B. K. P. (1986), “Robot Vision”, McGraw-Hill Company, pp. 278-298.
Intengan H. D., He G., Schiffrin E. L. (1998a), “Effect of vasopressin antagonism on structure and mechanics of small arteries and vascular expression of endothelin-1 in deoxycorticosterone acetate-salt hypertensive rats”, Hypertension, Vol. 32, pp. 770-777.
Intengan H. D., Schiffrin E. L. (1998b), “Mechanical properties of mesenteric resistance arteries from Dahl salt-sensitive rats: role of endothelin-1”, Journal of Hypertension, Vol. 16, pp. 1907-1912.
Johns D. G., Jin J-S, Webb R. C. (1998), “The role of the endothelium in ceramide-induced vasodilation”, European Journal of Pharmacology, Vol. 349, pp. R9-R10.
Kanai H., Hasegawa H., Ichiki M., Tezuka F., Koiwa Y. (2003), “Elasticity imaging of atheroma with transcutaneous ultrasound: Preliminary study”, Circulation, Vol. 107, No. 24, pp. 3018-3021.
Le Mehaute B. An introduction to hydrodynamics and water waves. New-York: Springer-Verlag, 1976, pp. 18-20.
Lee; R. T., Loree, H. M., Cheng, G. C., Lieberman, E. H., Jaramillo, N., Schoen, F. J. (1993), “Computational structural analysis based on intravascular ultrasound imaging before in vitro angioplasty: prediction of plaque fracture locations,” J. Am. Coll. Cardiol., Vol. 21, No. 3, pp. 777-782.
Levenberg, K. (1963). “A method for the solution of certain non-linear problems in least-squares,” Q. Appl. Math., Vol. 2, No. 2, pp. 164-168.
Loree, H. M, Kamm, R. D., Stringfellow, R. G., Lee, R. T. (1992), “Effects of fibrous cap thickness on peak circumferential stress in model atherosclerotic vessels,” Circ Res, Vol. 71, No. 4, pp. 850-858.
Mai J. J., Insana M. (2002), “Strain imaging of internal deformation”, Ultrasound in Med. & Biol., Vol. 28, Nos. 11/12, pp. 1475-1484.
Marquardt, D. W. (1944). “An algorithm for least-squares estimation of non-linear parameters,” J. SIAM, Vol. 11, No. 2, pp. 431-441.
Mase G. E. Theory and problems of continuum mechanics. Schaum's Outline Series, McGraw-Hill, New York, 1970.
Maurice R. L. and Bertrand M. (1999), “Lagrangian speckle model and tissue motion estimation—Theory,” IEEE Transactions on Medical Imaging, Vol. 18, No. 7, pp. 593-603.
Ohayon J., Teppaz P., Finet G., Rioufol G. (2001), “In-vivo prediction of human coronary plaque rupture location using intravascular ultrasound and the finite element method”, Coronary Artery Disease, Vol. 12, pp. 655-663.
Ophir J., Céspedes E. I., Ponnekanti H., Yazdi Y., and Li X. (1991), “Elastography: a quantitative method for imaging the elasticity in biological tissues”, Ultrasonic Imaging, Vol. 13, No. 2, pp. 111-134.
Ophir J., Alam S. K., Garra B., Kallel F., Konofagou E., Krouskop T., and Varghese T. (1999), “Elastography: ultrasonic estimation and imaging of the elastic properties of tissues,” Proceedings of the Institution of Mechanical Engineers. Part H—Journal of Engineering in Medicine, Vol. 213, No. H3, pp. 203-233.
Ryan L. K. and Foster F. S. (1997), “Ultrasonic measurement of differential displacement strain in a vascular model,” Ultrasonic Imaging, Vol. 19, No. 1, pp. 19-38.
Shapo B. M., Crowe J. R., Skovoroda A. R., Eberle M. J., Cohn N. A., O'Donnell M. (1996a), “Displacement and strain imaging of coronary arteries with intraluminal ultrasound”, IEEE Transactions on Ultrasonics, Ferroelectrics, & Frequency Control, Vol. 43(2), pp. 234-246.
Shapo B. M., Crowe J. R., Erkamp R., Emelianov S. Y., Eberle M. J., O'Donnell M. (1996b), “Strain imaging of coronary arteries with intraluminal ultrasound: Experiments on an inhomogeneous phantom”, Ultrasonic Imaging, Vol. 18(3), pp. 173-191.
Si X., Webb R. C., Richey J. M. (1999), “Bezafibrate, an anti-hypertriglyceridemic drug, attenuates vascular hyperresponsiveness and elevated blood pressure in fructose-induced hypertensive rats”, Can. J. Physiol. Pharmacol., Vol. 77, pp. 755-762.
Tatchum-Talom R., Martel C., Marette A. (2001), “Influence of estrogen on aortic stiffness and endothelial function in female rats”, Am. J. Physiol., Heart Circ. Physiol., Vol. 282, pp. H491-H498.

Talhami H. E., Wilson L. S., Neale M. L. (1994), “Spectral tissue strain: A new technique for imaging tissue strain using intravascular ultrasound”, Ultrasound in Medicine & Biology, Vol. 20(8), pp. 759-772.
Treyve, F., Ohayon, J., Finet, G., and Rioufol, G. (2003). “Modelling of the Stress distribution in an atherosclerotic plaque in man using a finite element analysis,” C. R. Mécanique, Vol. 331, pp. 449-454.
Weitzel W.F., Kim K., Rubin J. M., Wiggins R. C., Xie H., Chen X., Emelianov S. Y., O'Donnell M., 2004, “Feasibility of applying ultrasound strain imaging to detect renal transplant chronic allograft nephropathy”, Kidney Int., Vol. 65, No. 2, pp. 733-736.
Williamson, S. D., Lam, Y., Younis, H. F., Huang, H., Patel, S., Kaazempur-Mofrad, M. R., and Kamm, R. D. (2003), “On the sensitivity of wall stresses in diseased arteries to variable material properties,”Journal of Biomechanical Engineering, Vol. 125, pp. 147-155.
Zaman A. G., Helft G., Worthley S. G., Badimon J. J. (2000), “The role of plaque rupture in coronary artery disease”, Atherosclerosis, Vol. 149, pp. 251-266.
Zhao J., Day J., Yuan Z. F., Gregersen H. (2002), “Regional artery stress-strain distributions referenced to the zero-stress state in the rat”, Am. J. Physiol., Heart Circ. Physiol., Vol. 282, pp. H622-H629.

What is claimed is:

1. A method for vascular elastography comprising:
providing pre-tissue-motion and post-tissue-motion images in digital form of a vessel delimited by a vascular wall; said pre-tissue-motion and post-tissue-motion images being representative of first and second time-delayed configuration of said vessel;
partitioning at least portions of both said pre-tissue-motion and post-tissue-motion images into corresponding data windows;
approximating a trajectory between said pre-tissue-motion and post-tissue-motion images for corresponding data windows; and
using the trajectory for each data window to compute a strain tensor in each data window.

2. A method as recited in claim 1, further comprising using said strain tensor in each data window to create an elastogram of at least part of said vessel.

3. A method as recited in claim 1, wherein said pre-tissue-motion and post-tissue-motion images are radio-frequency (RF) images.

4. A method as recited in claim 3, wherein said pre-tissue-motion and post-tissue-motion images are part of a sequence of radio-frequency (RF) images.

5. A method as recited in claim 1, wherein said pre-tissue-motion and post-tissue-motion images are issued from magnetic resonance imaging (MRI), optical coherence tomography (OCT), brightness mode (B-mode) or Doppler-based ultrasound modality imaging.

6. A method as recited in claim 1, wherein providing pre-tissue-motion and post-tissue-motion images in digital form of a vessel includes inducing tissue compression or dilatation on said vessel.

7. A method as recited in claim 6, wherein inducing tissue dilatation on said vessel is achieved by cardiac pulsation.

8. A method as recited in claim 1, wherein said strain tensor is the full strain tensor in at least one of said data windows.

9. A method as recited in claim 8, wherein said full strain tensor is computed from three-dimensional or two-dimensional ultrasound data.

10. A method as recited in claim 8, further comprising using said full strain tensor to compute the Von Mises (VM) coefficient in each data window.

11. A method as recited in claim 1, wherein approximating a trajectory for each said data window includes using a Lagrangian speckle model estimator (LSME).

12. A method as recited in claim 1, wherein a trajectory is approximated in each said data window using zero-order and first-order terms of a Taylor-series expansion, yielding:
$$[ \begin{matrix}x \\ y \\ z\end{matrix} ] = \underbrace{[ \begin{matrix}x ( 0 , 0 , 0 , t ) \\ y ( 0 , 0 , 0 , t ) \\ z ( 0 , 0 , 0 , t )\end{matrix} ]}_{Tr} + \underbrace{{[ \begin{matrix}\frac{\partial x}{\partial {x}_{0}} & \frac{\partial x}{\partial {y}_{0}} & \frac{\partial x}{\partial {z}_{0}} \\ \frac{\partial y}{\partial {x}_{0}} & \frac{\partial y}{\partial {y}_{0}} & \frac{\partial y}{\partial {z}_{0}} \\ \frac{\partial z}{\partial {x}_{0}} & \frac{\partial z}{\partial {y}_{0}} & \frac{\partial z}{\partial {z}_{0}}\end{matrix} ]}_{( 0 , 0 , 0 , t )}}_{LT} [ \begin{matrix}{x}_{0} \\ {y}_{0} \\ {z}_{0}\end{matrix} ]$$
[Tr] is a translation vector
[LT] is a linear geometrical transformation of coordinates (x, y, z) represents the new position of a point (x₀, Y₀, z₀).

13. A method as recited in claim 12, wherein using said trajectory for each said data window to compute a strain tensor in each data window includes performing a non-linear minimization for each data window Wᵢⱼ by computing a transformation [LT] providing the best match between each Wᵢⱼ of said pre-tissue motion image and a corresponding window Wᵢⱼ in said post-tissue motion image.

14. A method as recited in claim 12, further comprising computing the full strain tensor ε having the components εᵢⱼ wherein:
$${ɛ}_{ij} ( t ) = \frac{1}{2} [ {Δ}_{ij} ( t ) + {Δ}_{ij} ( t ) ] \\ [ \begin{matrix}u \\ v \\ w\end{matrix} ] = [ \begin{matrix}x - {x}_{0} \\ y - {y}_{0} \\ z - {z}_{0}\end{matrix} ] = \underbrace{[ \begin{matrix}x ( 0 , 0 , 0 , t ) \\ y ( 0 , 0 , 0 , t ) \\ z ( 0 , 0 , 0 , t )\end{matrix} ]}_{Tr} + Δ [ \begin{matrix}{x}_{0} \\ {y}_{0} \\ {z}_{0}\end{matrix} ] , with \text{:}$$
$$Δ = \underbrace{{[ \begin{matrix}\frac{\partial x}{\partial {x}_{0}} - 1 & \frac{\partial x}{\partial {y}_{0}} & \frac{\partial x}{\partial {z}_{0}} \\ \frac{\partial y}{\partial {x}_{0}} & \frac{\partial y}{\partial {y}_{0}} - 1 & \frac{\partial y}{\partial {z}_{0}} \\ \frac{\partial z}{\partial {x}_{0}} & \frac{\partial z}{\partial {y}_{0}} & \frac{\partial z}{\partial {z}_{0}} - 1\end{matrix} ]}_{( 0 , 0 , 0 , t )}}_{LT - I}$$
(u, v, w) being displacement vector in the Cartesian coordinate system.

15. A method as recited in claim 14, further comprising determining an elastogram providing a distribution of each component of the deformation matrix Δ and of the strain tensor ε.

16. A method as recited in claim 15, further comprising computing a pressure gradient between said pre-tissue-motion and post-tissue-motion images; said pressure gradient being used in determining said elastogram.

17. A method as recited in claim 15, further comprising computing the Von Mises (VM) ξ coefficient in at least some of said data windows as:
$$ξ = {\{ \frac{2}{9} [ \begin{matrix}{( {ɛ}_{xx} - {ɛ}_{yy} )}^{2} + {( {ɛ}_{yy} - {ɛ}_{zz} )}^{2} + \\ {( {ɛ}_{zz} - {ɛ}_{xx} )}^{2} + 6 ( {ɛ}_{xy}^{2} + {ɛ}_{yz}^{2} + {ɛ}_{xz}^{2} )\end{matrix} ] \}}^{1 / 2}$$

18. A method as recited in claim 17, further comprising determining a composite elastogram providing a distribution of the VM coefficient in at least some of said data windows.

19. A method as recited in claim 17, further comprising:
providing pressure gradient σ resulting from blood flow pulsation of said vessel when said pre-tissue motion and post-tissue motion images are taken; and
computing the elastic modulus in at least some of said data windows as:
$$E = \frac{σ}{ξ} .$$

20. A method as recited in claim 12, wherein using said trajectory for each said data window to compute a strain tensor in each data window includes solving the following minimization equation:
$$\begin{matrix}MIN \\ {Ψ}_{ij}\end{matrix} {‖ I ( x ( {t}_{0} ) , y ( {t}_{0} ) , z ( {t}_{0} ) ) - {I}_{Lag} ( \begin{matrix}x ( {t}_{0} + Δ t ) , y ( {t}_{0} + Δ t ) , \\ z ( {t}_{0} + Δ t )\end{matrix} ) ‖}^{2}$$
where ψᵢⱼ=[Tr;LT(:)] for data window Wᵢⱼ for augmented vector (;) and matrix vectorisation (:)
I_{Lag}(x(t₀+Δt),y(t₀+Δt),z(t₀+Δt)) is the Lagrangian speckle image (LSI) defined as the post-tissue motion image I(x(t₀+Δt),y(t₀+Δt),z(t₀+Δt)) numerically compensated for tissue motion.

21. A method as recited in claim 20, wherein solving said minimization equation includes using a minimization algorithm.

22. A method as recited in claim 21, wherein said minimization algorithm is the regularized nonlinear minimization Levenberg-Marquardt (L&M) minimization algorithm.

23. A method as recited in claim 12, wherein using said trajectory for each said data window to compute a strain tensor in each data window includes solving in a region of interest represented in both said pre-tissue-motion and post-tissue-motion images characterized by p×q pixels:
$$[ \begin{matrix}{I}_{{x}_{1}} {x}_{1} & {I}_{{x}_{1}} {y}_{1} & {I}_{{x}_{1}} {z}_{1} & {I}_{{x}_{1}} & ⋯ & {I}_{{z}_{1}} {x}_{1} & {I}_{{z}_{1}} {y}_{1} & {I}_{{z}_{1}} {z}_{1} & {I}_{{z}_{1}} \\ {I}_{{x}_{2}} {x}_{2} & {I}_{{x}_{2}} {y}_{2} & {I}_{{x}_{2}} {z}_{2} & {I}_{{x}_{2}} & ⋯ & {I}_{{z}_{2}} {x}_{2} & {I}_{{z}_{2}} {y}_{2} & {I}_{{z}_{2}} {z}_{2} & {I}_{{z}_{2}} \\ ⋮ & ⋮ & ⋮ & ⋮ & & ⋮ & ⋮ & ⋮ & ⋮ \\ & & & & & & & & \\ & & & & & & & & \\ & & & & & & & & \\ ⋮ & ⋮ & ⋮ & ⋮ & ⋯ & ⋮ & ⋮ & ⋮ & ⋮ \\ & & & & & & & & \\ & & & & & & & & \\ & & & & & & & & \\ ⋮ & ⋮ & ⋮ & ⋮ & ⋯ & ⋮ & ⋮ & ⋮ & ⋮ \\ {I}_{{x}_{p \times q}} {x}_{p \times q} & {I}_{{x}_{p \times q}} {y}_{p \times q} & {I}_{{x}_{p \times q}} {z}_{p \times q} & {I}_{{x}_{p \times q}} & ⋯ & {I}_{{z}_{p \times q}} {x}_{p \times q} & {I}_{{z}_{p \times q}} {y}_{p \times q} & {I}_{{z}_{p \times q}} {z}_{p \times q} & {I}_{{z}_{p \times q}}\end{matrix} ] [ \begin{matrix}{Δ}_{xx} \\ {Δ}_{xy} \\ {Δ}_{xz} \\ {t}_{x} \\ {Δ}_{yx} \\ {Δ}_{yy} \\ {Δ}_{yz} \\ {t}_{y} \\ {Δ}_{zx} \\ {Δ}_{zy} \\ {Δ}_{zz} \\ {t}_{z}\end{matrix} ] = [ \begin{matrix}{\tilde{I}}_{{t}_{1}} \\ {\tilde{I}}_{{t}_{2}} \\ ⋮ \\ \\ \\ \\ ⋮ \\ \\ \\ \\ ⋮ \\ {\tilde{I}}_{{t}_{p \times q}}\end{matrix} ] .$$
for each corresponding said pixels in said pre-tissue motion and post-tissue motion images in digital form;
where tₓ=x(0,0,0,t); t_{y}=y(0,0,0,t); t_{z}=z(0,0,0,t); and Ĩₜ=(I_{Lag}(x(t+dt),y(t+dt),z(t+dt))−I(x(t),y(t),z(t))).

24. A method as recited in claim 1, wherein providing pre-tissue motion and post-tissue motion images in digital form includes collecting longitudinal and cross-sectional radio-frequency (RF) data from said vessel.

25. A method recited in claim 1 for endovascular elastography (EVE).

26. A method as recited in claim 24, wherein providing pre-tissue-motion and post-tissue motion images includes acquiring intravascular RF images using a catheter.

27. A method as recited in claim 26, wherein acquiring intravascular RF images using a catheter includes sequentially sweeping an ultrasound beam over a predetermined angle.

28. A method as recited in claim 1 for non-invasive vascular elastography (NIVE).

29. A method as recited in claim 25 for non-invasive microvascular elastography (MicroNIVE).

30. The use of the method from claim 1 for predicting risks of vascular tissue rupture or vascular aneurysms.

31. The use of the method from claim 1 for phenotyping in animal models using genetic or cloning technologies.

32. The use as recited in claim 31 wherein said model is hypertension (HT).

33. The use of the method from claim 1 for in vivo measurements.

34. A system for vascular elastography comprising:
an ultrasound system for acquiring pre-tissue motion and post-tissue motion radio-frequency (RF) images of a vessel; said pre-tissue motion and post-tissue motion images being representative of first and second time-delayed configuration of said vessel;
a controller, coupled to said ultrasound system, i) for receiving said pre-tissue motion and post-tissue motion RF images, ii) for digitizing said pre-tissue motion and post-tissue motion RF images, iii) for partitioning both said pre-tissue motion and post-tissue motion RF images into corresponding data windows, iv) for approximating a trajectory for each said data windows; and v) for using said trajectory for each said data window to compute a strain tensor in each data window; and
an output device coupled to said controller to output information related to said strain tensor in each data window.

35. A system as recited in claim 34, wherein said controller further includes an analog-to-digital acquisition board for digitizing said pre-tissue motion and post-tissue motion images.

36. A system as recited in claim 34, wherein said ultrasound system includes an ultrasound instrument, coupled to said analog-to-digital acquisition board.

37. A system as recited in claim 36, wherein said ultrasound instrument includes a scanhead.

38. A system as recited in claim 37, wherein said scanhead includes an array ultrasound transducer.

39. A system as recited in claim 37, wherein said scanhead includes a single-element oscillating transducer.

40. A system as recited in claim 36, wherein said ultrasound instrument includes a catheter having a tip and a transducer provided at said tip.

41. A system as recited in claim 36, wherein said ultrasound instrument is in the form of an ultrasound biomicroscope for non-invasive microvascular elastography (MicroNIVE) measurement.

42. A system as recited in claim 36, wherein said ultrasound instrument is coupled to said analog-to-digital acquisition board via a radio-frequency (RF) pre-amplifier.

43. The use of the system recited in claim 34 for predicting risks of vascular tissue rupture or vascular aneurysms.

44. The use of the system from claim 32 for phenotyping in animal models using genetic or cloning technologies.

45. The use of the system from claim 44 wherein said model is hypertension (HT).

46. A system for vascular elastography comprising:
means for providing pre-tissue motion and post-tissue motion images in digital form of a vessel; said pre-tissue motion and post-tissue motion images being representative of first and second time-delayed configuration of said vascular vessel;
means for partitioning both said pre-tissue motion and post-tissue motion images into corresponding data windows;
means for approximating a trajectory for each said data windows; and
means for computing a strain tensor in each data window using said trajectory for each said data window.
